SEQUENCE LISTING (70 sequences)

SEQ ID NO: 1 - DNA, 46 bp, synthetic construct
  source 1..46: mol_type = other DNA
cacacaggaaacagctatgaccatgcttcaggtggctcgagtagtg

SEQ ID NO: 2 - DNA, 45 bp, synthetic construct
  source 1..45: mol_type = other DNA
cacacaggaaacagctatgaccatgtttcttttgcctgcaggatt

SEQ ID NO: 3 - DNA, 48 bp, synthetic construct
  source 1..48: mol_type = other DNA
cacacaggaaacagctatgaccatgtggacaatgtggagataaaggac

SEQ ID NO: 4 - DNA, 43 bp, synthetic construct
  source 1..43: mol_type = other DNA
cacacaggaaacagctatgaccatgctggtcctgcctcactcg

SEQ ID NO: 5 - DNA, 45 bp, synthetic construct
  source 1..45: mol_type = other DNA
cacacaggaaacagctatgaccatggaagcaaacagctcgcaagt

SEQ ID NO: 6 - DNA, 44 bp, synthetic construct
  source 1..44: mol_type = other DNA
cacacaggaaacagctatgaccatggccagctgttgctgaccta

SEQ ID NO: 7 - DNA, 45 bp, synthetic construct
  source 1..45: mol_type = other DNA
cacacaggaaacagctatgaccatgagacctcaggcggctcatag

SEQ ID NO: 8 - DNA, 45 bp, synthetic construct
  source 1..45: mol_type = other DNA
cacacaggaaacagctatgaccatggagtggttggggaggacatt

SEQ ID NO: 9 - DNA, 43 bp, synthetic construct
  source 1..43: mol_type = other DNA
cacacaggaaacagctatgaccatgctgtcaaggcgggcttct

SEQ ID NO: 10 - DNA, 45 bp, synthetic construct
  source 1..45: mol_type = other DNA
cacacaggaaacagctatgaccatgatcgggcagtgatagagcag

SEQ ID NO: 11 - DNA, 45 bp, synthetic construct
  source 1..45: mol_type = other DNA
cacacaggaaacagctatgaccatggaagcctctgattggcacag

SEQ ID NO: 12 - DNA, 55 bp, synthetic construct
  source 1..55: mol_type = other DNA
cacacaggaaacagctatgaccatggcttgtgagtggatgggtaaaacctatcat

SEQ ID NO: 13 - DNA, 55 bp, synthetic construct; identical to SEQ ID NO: 12
  source 1..55: mol_type = other DNA

SEQ ID NO: 14 - DNA, 53 bp, synthetic construct
  source 1..53: mol_type = other DNA
cacacaggaaacagctatgaccatgtccactacaactacatgtgtaacagttc

SEQ ID NO: 15 - DNA, 42 bp, synthetic construct
  source 1..42: mol_type = other DNA
cacacaggaaacagctatgaccatggcggaaaggaaggggag

SEQ ID NO: 16 - DNA, 42 bp, synthetic construct; identical to SEQ ID NO: 15
  source 1..42: mol_type = other DNA

SEQ ID NO: 17 - DNA, 47 bp, synthetic construct
  source 1..47: mol_type = other DNA
cacacaggaaacagctatgaccatgtggctctgactgtaccaccatc

SEQ ID NO: 18 - DNA, 44 bp, synthetic construct
  source 1..44: mol_type = other DNA
cacacaggaaacagctatgaccatgcggaaatgtgggaggagag

SEQ ID NO: 19 - DNA, 45 bp, synthetic construct
  source 1..45: mol_type = other DNA
cacacaggaaacagctatgaccatgccagcacctatggcaagttt

SEQ ID NO: 20 - DNA, 47 bp, synthetic construct
  source 1..47: mol_type = other DNA
cacacaggaaacagctatgaccatggccctgactttcaactctgtct

SEQ ID NO: 21 - DNA, 45 bp, synthetic construct
  source 1..45: mol_type = other DNA
cacacaggaaacagctatgaccatggtccaacgtgccatatccat

SEQ ID NO: 22 - DNA, 44 bp, synthetic construct
  source 1..44: mol_type = other DNA
cacacaggaaacagctatgaccatgccttgtggcaccctaggtc

SEQ ID NO: 23 - DNA, 42 bp, synthetic construct; identical to SEQ ID NO: 15
  source 1..42: mol_type = other DNA

SEQ ID NO: 24 - DNA, 42 bp, synthetic construct; identical to SEQ ID NO: 15
  source 1..42: mol_type = other DNA

SEQ ID NO: 25 - DNA, 42 bp, synthetic construct; identical to SEQ ID NO: 15
  source 1..42: mol_type = other DNA

SEQ ID NO: 26 - DNA, 42 bp, synthetic construct; identical to SEQ ID NO: 15
  source 1..42: mol_type = other DNA

SEQ ID NO: 27 - DNA, 48 bp, synthetic construct
  source 1..48: mol_type = other DNA
cacacaggaaacagctatgaccatggcaattcactgtaaagctggaaa

SEQ ID NO: 28 - DNA, 46 bp, synthetic construct
  source 1..46: mol_type = other DNA
cacacaggaaacagctatgaccatgtctgacataaaccaggccaac

SEQ ID NO: 29 - DNA, 44 bp, synthetic construct
  source 1..44: mol_type = other DNA
cacacaggaaacagctatgaccatgccgagtcctgggagttttg

SEQ ID NO: 30 - DNA, 45 bp, synthetic construct
  source 1..45: mol_type = other DNA
cacacaggaaacagctatgaccatgcgaggcactgcggtattaag

SEQ ID NO: 31 - DNA, 45 bp, synthetic construct
  source 1..45: mol_type = other DNA
cacacaggaaacagctatgaccatgacagccagtcgactctgagg

SEQ ID NO: 32 - DNA, 49 bp, synthetic construct
  source 1..49: mol_type = other DNA
cacacaggaaacagctatgaccatgaatggaacaaacaatgataagcaa

SEQ ID NO: 33 - DNA, 53 bp, synthetic construct; identical to SEQ ID NO: 14
  source 1..53: mol_type = other DNA

SEQ ID NO: 34 - DNA, 55 bp, synthetic construct
  source 1..55: mol_type = other DNA
cacacaggaaacagctatgaccatgactcacatgttgatttgacttactaatgta

SEQ ID NO: 35 - DNA, 52 bp, synthetic construct
  source 1..52: mol_type = other DNA
cgacgtaaaacgacggccagtnnnnnnnnnnnnnngtgaggggccgtgttgt

SEQ ID NO: 36 - DNA, 57 bp, synthetic construct
  source 1..57: mol_type = other DNA
cgacgtaaaacgacggccagtnnnnnnnnnnnnnncctgaattgtagcaatcaccaa

SEQ ID NO: 37 - DNA, 58 bp, synthetic construct
  source 1..58: mol_type = other DNA
cgacgtaaaacgacggccagtnnnnnnnnnnnnnnccaccagtggaaataagaacaga

SEQ ID NO: 38 - DNA, 55 bp, synthetic construct
  source 1..55: mol_type = other DNA
cgacgtaaaacgacggccagtnnnnnnnnnnnnnntccctcccctagaaggtctg

SEQ ID NO: 39 - DNA, 54 bp, synthetic construct
  source 1..54: mol_type = other DNA
cgacgtaaaacgacggccagtnnnnnnnnnnnnnngcggtagcgatgaggtttc

SEQ ID NO: 40 - DNA, 56 bp, synthetic construct
  source 1..56: mol_type = other DNA
cgacgtaaaacgacggccagtnnnnnnnnnnnnnnagagtcctttctcctgctgct

SEQ ID NO: 41 - DNA, 55 bp, synthetic construct
  source 1..55: mol_type = other DNA
cgacgtaaaacgacggccagtnnnnnnnnnnnnnnttgcgtgtggagtatttgga

SEQ ID NO: 42 - DNA, 55 bp, synthetic construct
  source 1..55: mol_type = other DNA
cgacgtaaaacgacggccagtnnnnnnnnnnnnnngttccccatttgaggtctcg

SEQ ID NO: 43 - DNA, 52 bp, synthetic construct
  source 1..52: mol_type = other DNA
cgacgtaaaacgacggccagtnnnnnnnnnnnnnngcagcacgagggtcagg

SEQ ID NO: 44 - DNA, 55 bp, synthetic construct
  source 1..55: mol_type = other DNA
cgacgtaaaacgacggccagtnnnnnnnnnnnnnntcattgggtgttacgcttcc

SEQ ID NO: 45 - DNA, 55 bp, synthetic construct
  source 1..55: mol_type = other DNA
cgacgtaaaacgacggccagtnnnnnnnnnnnnnnagcaggcataggaacatcca

SEQ ID NO: 46 - DNA, 65 bp, synthetic construct
  source 1..65: mol_type = other DNA
cgacgtaaaacgacggccagtnnnnnnnnnnnnnnatgcaaaatcacattattgccaacatgact

SEQ ID NO: 47 - DNA, 65 bp, synthetic construct; identical to SEQ ID NO: 46
  source 1..65: mol_type = other DNA

SEQ ID NO: 48 - DNA, 55 bp, synthetic construct
  source 1..55: mol_type = other DNA
cgacgtaaaacgacggccagtnnnnnnnnnnnnnntgtgatgatggtgaggatgg

SEQ ID NO: 49 - DNA, 50 bp, synthetic construct
  source 1..50: mol_type = other DNA
cgacgtaaaacgacggccagtnnnnnnnnnnnnnncccgtcccgacccct

SEQ ID NO: 50 - DNA, 50 bp, synthetic construct; identical to SEQ ID NO: 49
  source 1..50: mol_type = other DNA

SEQ ID NO: 51 - DNA, 53 bp, synthetic construct
  source 1..53: mol_type = other DNA
cgacgtaaaacgacggccagtnnnnnnnnnnnnnngtggcaagtggctcctga

SEQ ID NO: 52 - DNA, 59 bp, synthetic construct
  source 1..59: mol_type = other DNA
cgacgtaaaacgacggccagtnnnnnnnnnnnnnnaaagcccataaaggaatgtaaacc

SEQ ID NO: 53 - DNA, 53 bp, synthetic construct
  source 1..53: mol_type = other DNA
cgacgtaaaacgacggccagtnnnnnnnnnnnnnncgggatggcttgaagaga

SEQ ID NO: 54 - DNA, 53 bp, synthetic construct
  source 1..53: mol_type = other DNA
cgacgtaaaacgacggccagtnnnnnnnnnnnnnngggggtgtggaatcaacc

SEQ ID NO: 55 - DNA, 53 bp, synthetic construct
  source 1..53: mol_type = other DNA
cgacgtaaaacgacggccagtnnnnnnnnnnnnnnggtggggagtgggactca

SEQ ID NO: 56 - DNA, 55 bp, synthetic construct
  source 1..55: mol_type = other DNA
cgacgtaaaacgacggccagtnnnnnnnnnnnnnngttcagcctcctccctcatc

SEQ ID NO: 57 - DNA, 56 bp, synthetic construct
  source 1..56: mol_type = other DNA
cgacgtaaaacgacggccagtnnnnnnnnnnnnnncatcagatacaacgccaccat

SEQ ID NO: 58 - DNA, 50 bp, synthetic construct; identical to SEQ ID NO: 49
  source 1..50: mol_type = other DNA

SEQ ID NO: 59 - DNA, 50 bp, synthetic construct; identical to SEQ ID NO: 49
  source 1..50: mol_type = other DNA

SEQ ID NO: 60 - DNA, 50 bp, synthetic construct; identical to SEQ ID NO: 49
  source 1..50: mol_type = other DNA

SEQ ID NO: 61 - DNA, 50 bp, synthetic construct; identical to SEQ ID NO: 49
  source 1..50: mol_type = other DNA

SEQ ID NO: 62 - DNA, 57 bp, synthetic construct
  source 1..57: mol_type = other DNA
cgacgtaaaacgacggccagtnnnnnnnnnnnnnntggtccttacttccccatagaa

SEQ ID NO: 63 - DNA, 56 bp, synthetic construct
  source 1..56: mol_type = other DNA
cgacgtaaaacgacggccagtnnnnnnnnnnnnnnaccaggtcttcgtaagcatga

SEQ ID NO: 64 - DNA, 55 bp, synthetic construct
  source 1..55: mol_type = other DNA
cgacgtaaaacgacggccagtnnnnnnnnnnnnnntacagctgaggggaggagaa

SEQ ID NO: 65 - DNA, 55 bp, synthetic construct
  source 1..55: mol_type = other DNA
cgacgtaaaacgacggccagtnnnnnnnnnnnnnntgggtcttcctcctcctctg

SEQ ID NO: 66 - DNA, 53 bp, synthetic construct
  source 1..53: mol_type = other DNA
cgacgtaaaacgacggccagtnnnnnnnnnnnnnnatctgactccgccgattg

SEQ ID NO: 67 - DNA, 55 bp, synthetic construct
  source 1..55: mol_type = other DNA
cgacgtaaaacgacggccagtnnnnnnnnnnnnnnggaaggggcgaaattacagt

SEQ ID NO: 68 - DNA, 55 bp, synthetic construct; identical to SEQ ID NO: 48
  source 1..55: mol_type = other DNA

SEQ ID NO: 69 - DNA, 58 bp, synthetic construct
  source 1..58: mol_type = other DNA
cgacgtaaaacgacggccagtnnnnnnnnnnnnnnacagtacaaaatggaggacctga

SEQ ID NO: 70 - DNA, 44 bp, synthetic construct
  source 1..44: mol_type = other DNA
cacacaggaaacagctatgaccatgggaagttgatccgctcaca

(19) United States
(12) Patent Application Publication
(10) Pub. No.: US 2025/0027167 A1
(43) Pub. Date: Jan. 23, 2025

(54) DETECTION OF TUMOR-DERIVED DNA IN CEREBROSPINAL FLUID

(71) Applicant: The Johns Hopkins University, Baltimore, MD (US)

(72) Inventors: Chetan Bettegowda, Lutherville, MD (US); Kenneth W. Kinzler, Frankford, DE (US); Bert Vogelstein, Baltimore, MD (US); Yuxuan Wang, Baltimore, MD (US); Luis Diaz, Ellicott City, MD (US); Nickolas Papadopoulos, Towson, MD (US)

(21) Appl. No.: 18/740,012

(22) Filed: Jun. 11, 2024

Related U.S. Application Data
(63) Continuation of application No. 15/744,299, filed on Jan. 12, 2018, now Pat. No. 12,054,781.
(60) Provisional application No. 62/192,424, filed on Jul. 14, 2015.

(51) Int. Cl. C12Q 1/6886

(52) U.S. Cl. CPC C12Q 1/6886; C12Q 2600/156

(57) ABSTRACT

As cell-free DNA from brain and spinal cord tumors cannot usually be detected in the blood, we assessed the cerebrospinal fluid (CSF) that bathes the CNS for tumor DNA, here termed CSF-tDNA. The results suggest that CSF-tDNA could be useful for the management of patients with primary tumors of the brain or spinal cord.

### CROSS-REFERENCE TO RELATED APPLICATIONS

This application is a continuation of U.S. application Ser. No. 15/744,299, filed on Jan. 12, 2018, which is a National Stage application under 35 U.S.C. § 371 of International Application No. PCT/US2016/041862, having an International Filing Date of Jul. 12, 2016, which claims the benefit of U.S. Provisional Application No. 62/192,424, filed on Jul. 14, 2015, each of which is incorporated by reference in its entirety.

This invention was made with government support under grant CA043460 awarded by the National Institutes of Health. The government has certain rights in the invention.

### SEQUENCE LISTING

This application contains a Sequence Listing that has been submitted electronically as an XML file named “44807-0136002_ST26_SL.XML.” The XML file, created on Jun. 5, 2024, is 64,615 bytes in size. The material in the XML file is hereby incorporated by reference in its entirety.

### TECHNICAL FIELD OF THE INVENTION

This invention is related to the area of nucleic acid assays. In particular, it relates to assays of small amounts of nucleic acid in body fluids.

### BACKGROUND OF THE INVENTION

Approximately 25,000 individuals each year are diagnosed with a malignant brain or spinal cord tumor in the United States and more than half of these patients will die from their disease (1). While there are a number of different subtypes of primary central nervous system (CNS) cancers, nearly all are treated with maximal safe surgical resection followed by radiation and, in some cases, chemotherapy. Given the lack of clinically available biomarkers for CNS malignancies, the conventional method for disease monitoring in these patients is radiographic, using either computed tomography (CT) or magnetic resonance imaging (MRI) (2). Unfortunately, anatomic changes detected by these imaging modalities are often non-specific and slow to change even in the face of progressing or regressing disease. Moreover, it can be difficult to discriminate between treatment effect and cancer growth with imaging alone (3). Patients must, therefore, have additional surgeries for definitive tissue diagnosis or inappropriately wait for radiographic findings to change as their disease progresses. As a result, there is a great need for more sensitive and specific tumor biomarkers in neuro-oncology. The recent success of detecting circulating tumor cells (CTC's) in the peripheral blood of glioblastoma patients represents an important step towards this goal, with reported sensitivities between 21% and 39% (4-6). Circulating tumor-derived DNA (ctDNA) is found in the plasma of patients with most forms of malignancies (7-11). However, brain tumors, including high-grade gliomas and medulloblastomas, are an exception, with only a minority giving rise to detectable levels of ctDNA, perhaps because of the blood-brain barrier (8).

There is a continuing need in the art to develop sensitive assays for difficult-to-detect analytes.

### SUMMARY OF THE INVENTION

According to one aspect of the invention a method is provided. Nucleic acids in a Cerebral Spinal Fluid (CSF) sample from a human with a central nervous system (CNS) cancer are assayed for one or more mutations in the nucleic acids. The mutations are present in the cancer but not in normal tissues of the human.

Another aspect of the invention is a method in which nucleic acids in a CNS cancer tissue of a human are assayed, and one or more mutations are determined. Nucleic acids in a normal tissue of the human are also assayed, and absence of the one or more mutations is determined. Nucleic acids in a Cerebral Spinal Fluid (CSF) sample from the human are assayed for the one or more mutations in the sample using a specific probe or specific primer to detect the one or more mutations. The specific probe or specific primer hybridizes at or within 200 nucleotides of the mutation.

These and other embodiments which will be apparent to those of skill in the art upon reading the specification provide the art with

### BRIEF DESCRIPTION OF THE DRAWINGS

FIG. 1. Schematic showing the shedding of CSF-tDNA from Central Nervous System (CNS) malignancies. Tumor cells from primary brain and spinal cord tumors shed DNA into the cerebrospinal fluid (CSF) that bathes the CNS. DNA purified from the CSF is analyzed for tumor-specific mutations.

FIGS. 2A-2B. Representative MR images. (FIG. 2A) Example of tumor (red arrow) abutting a CSF space is shown. (FIG. 2B) Example of tumor not in contact with a CSF space is shown. Corresponding T2 images are provided for easier visualization of CSF.

FIG. 3 (Table 1). Patient Demographics

FIG. 4 (Table 2). Mutations detected in the CSF and tumor of each patient

FIG. 5A-5B (Tables 3A-3B). FIG. 5A. Associations between clinical characteristics and levels of CSF-tDNA. FIG. 5B. Associations between clinical characteristic and levels of CSF-tDNA.

FIG. 6 (Table 4). Detection of CSF-tDNA using whole-exome sequencing (WWS)

FIG. 7 (Table S1). Primers used for Mutations Detection. Forward primers (SEQ ID NO: 1-35). Reverse Primers (SEQ ID NO: 36-70).

FIG. 8 (Table S1). Mutations identified in whole-exome sequencing

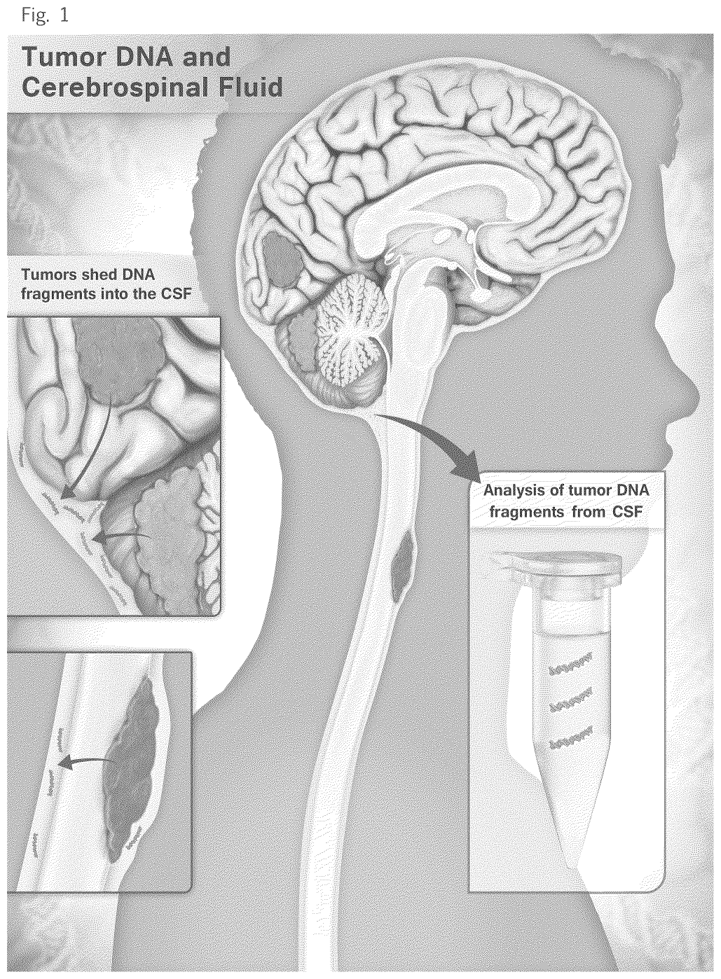

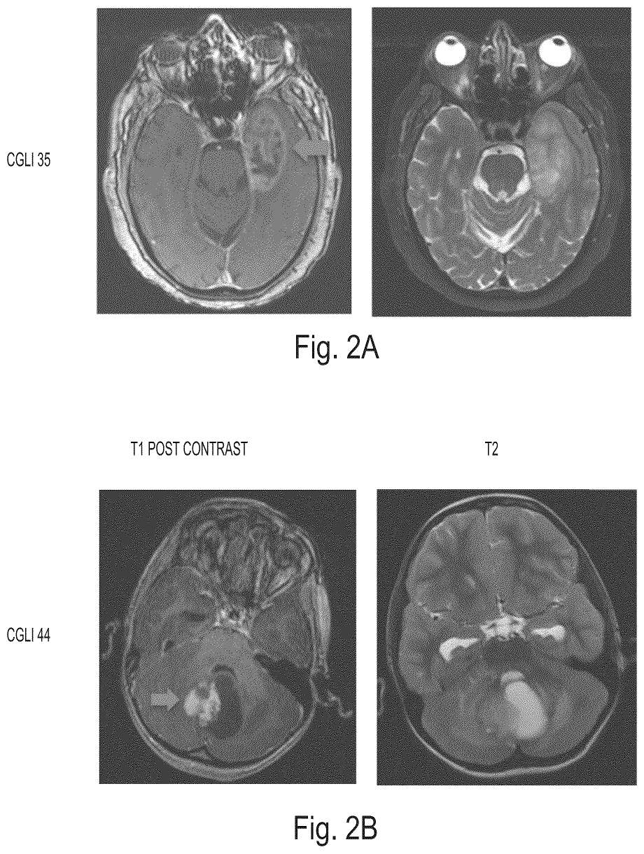

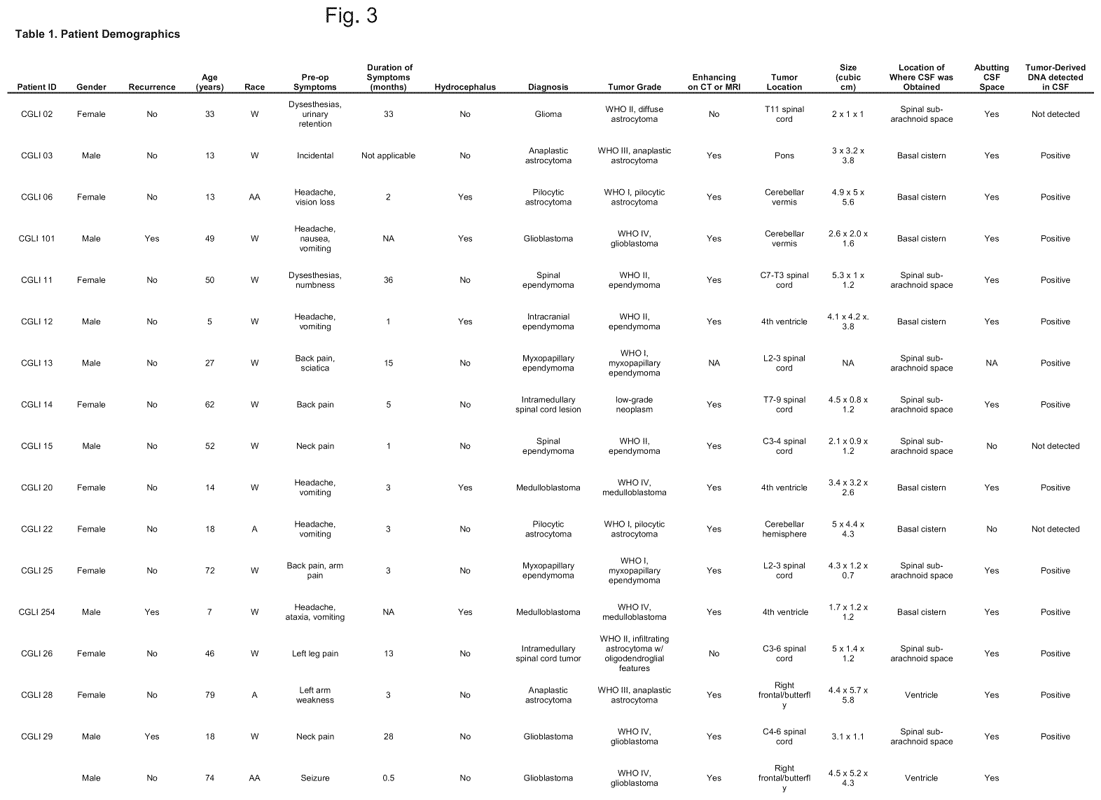

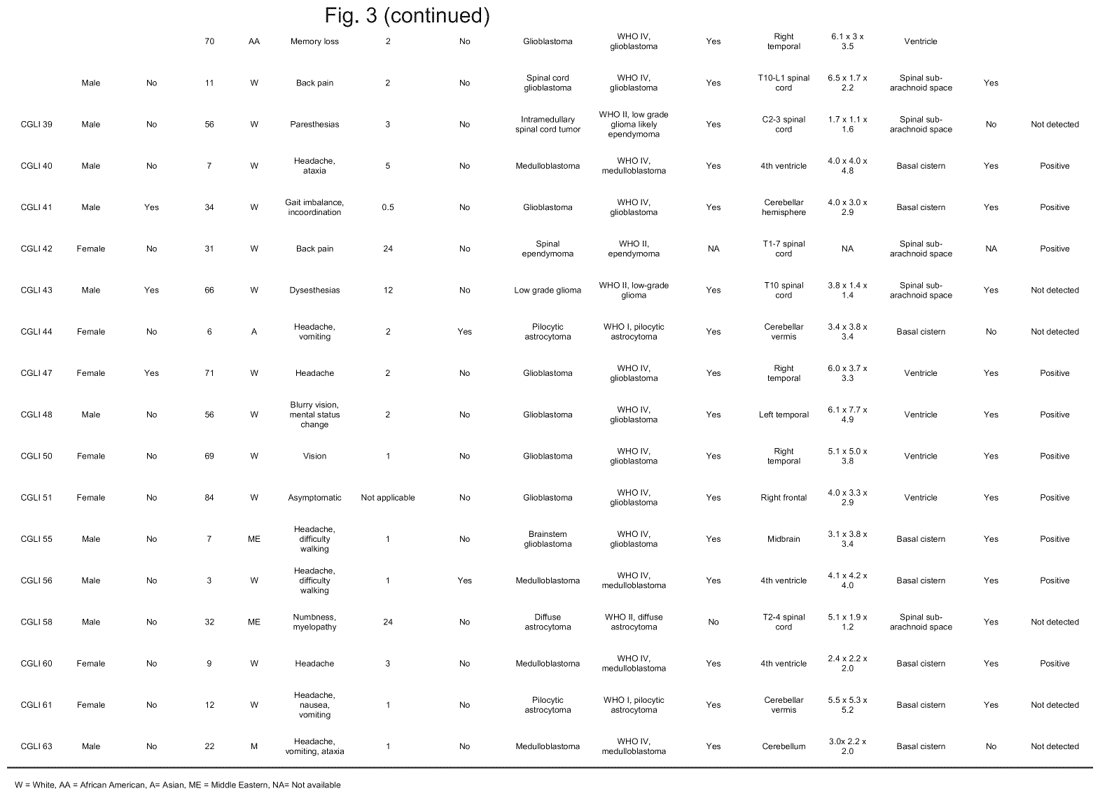

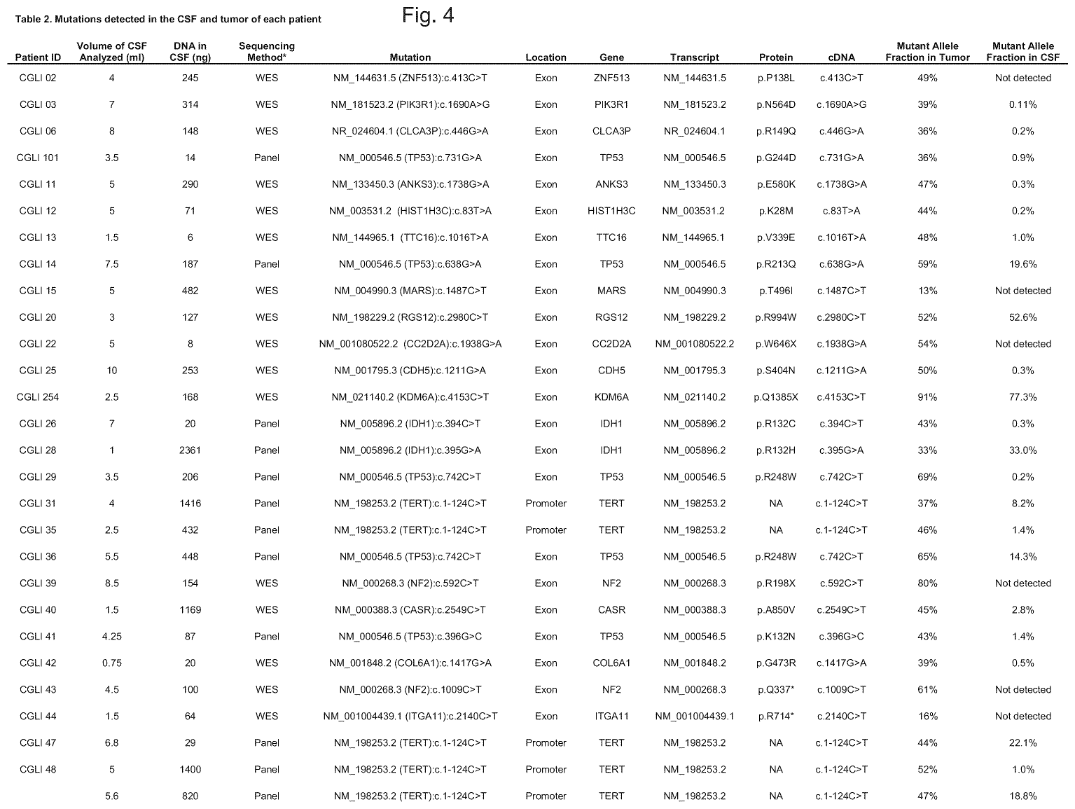

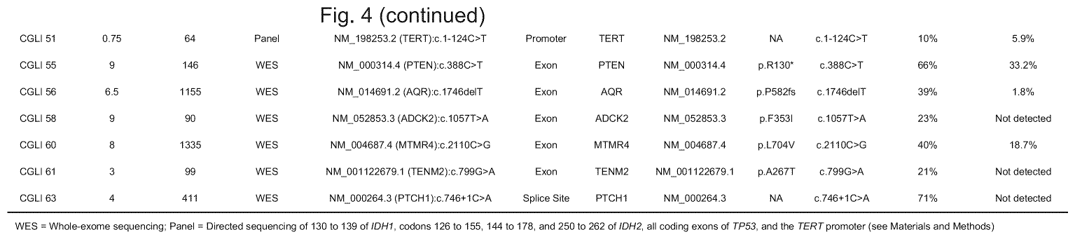

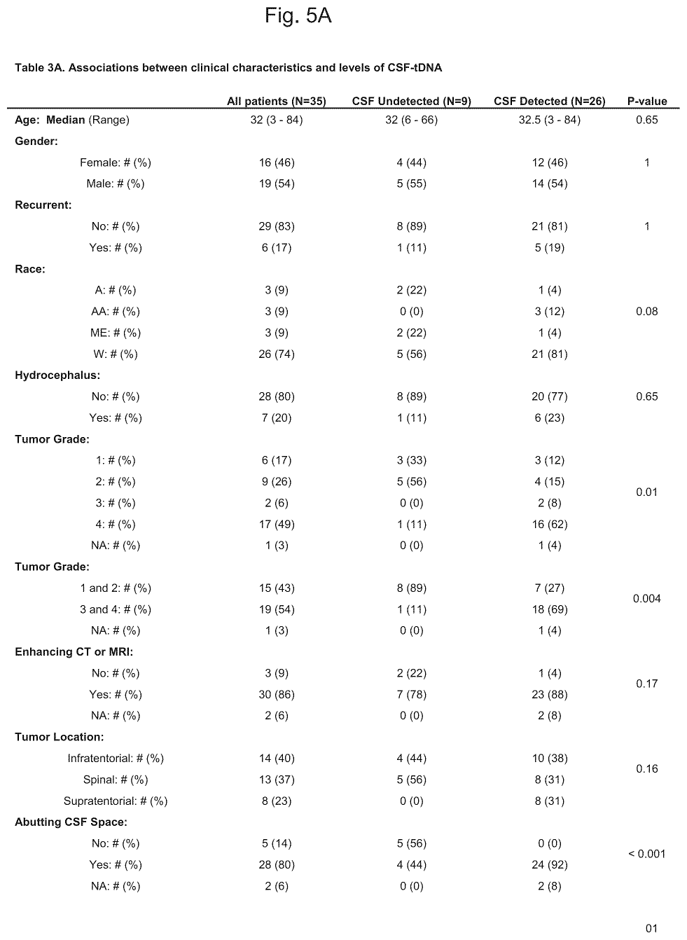

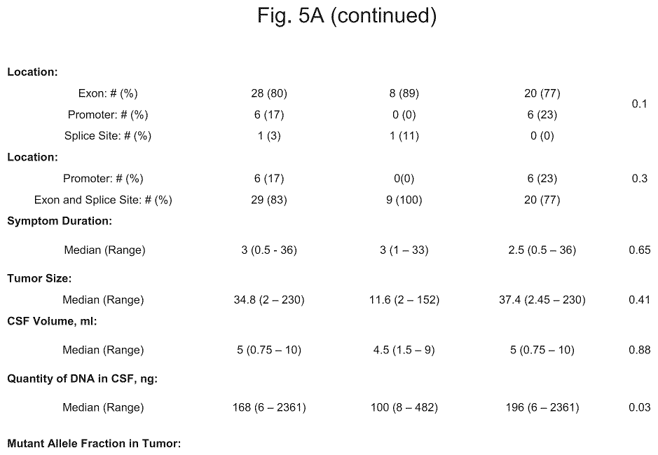

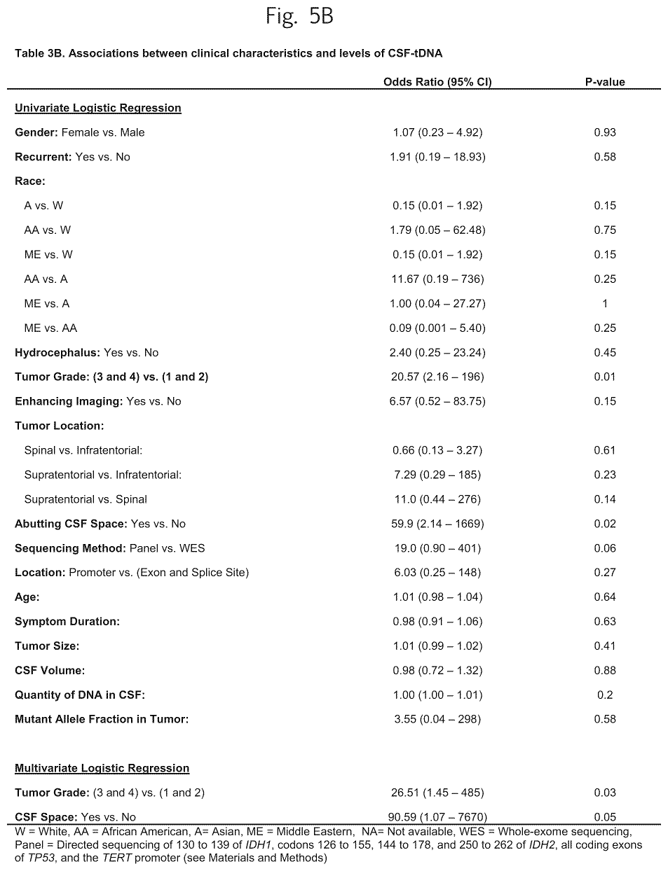

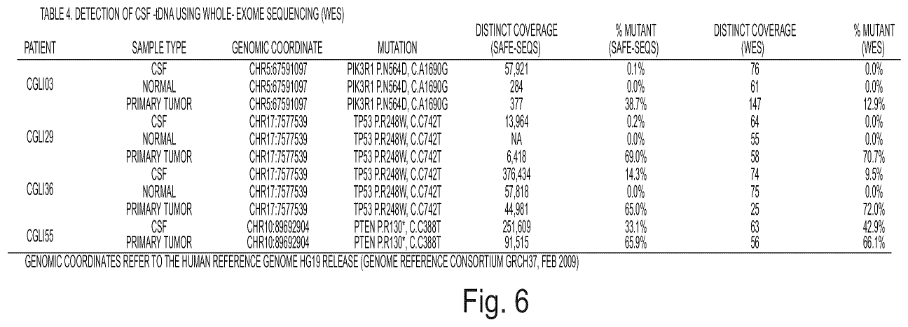

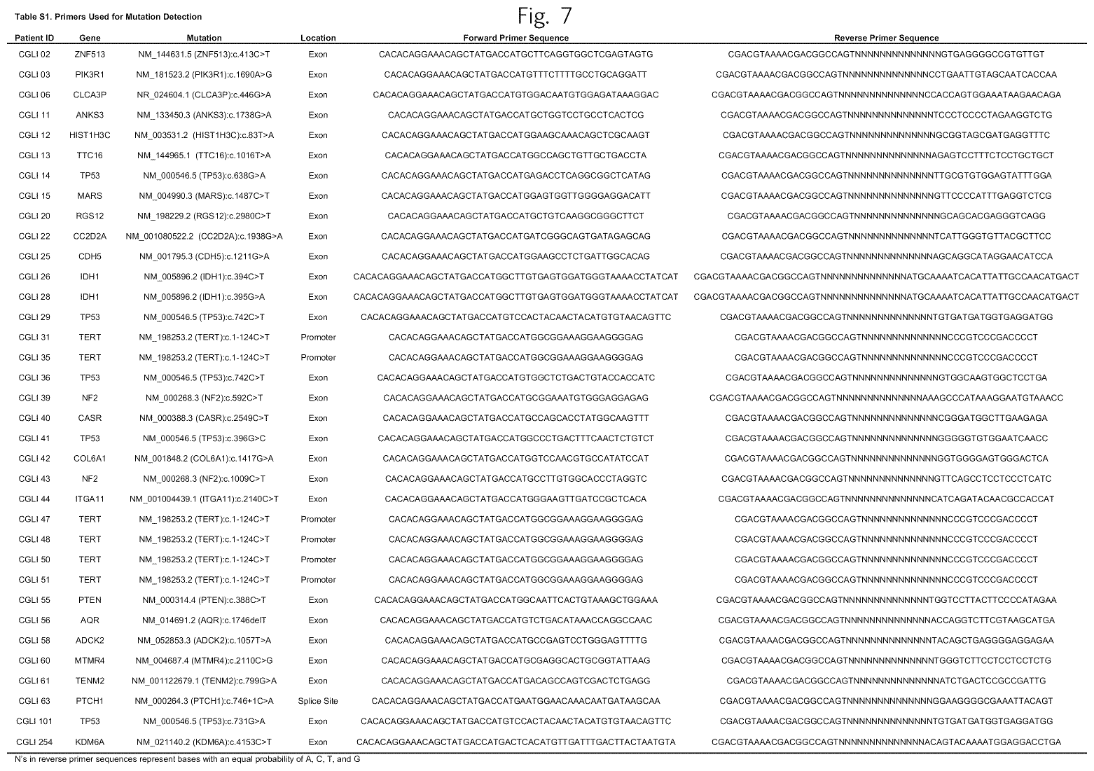

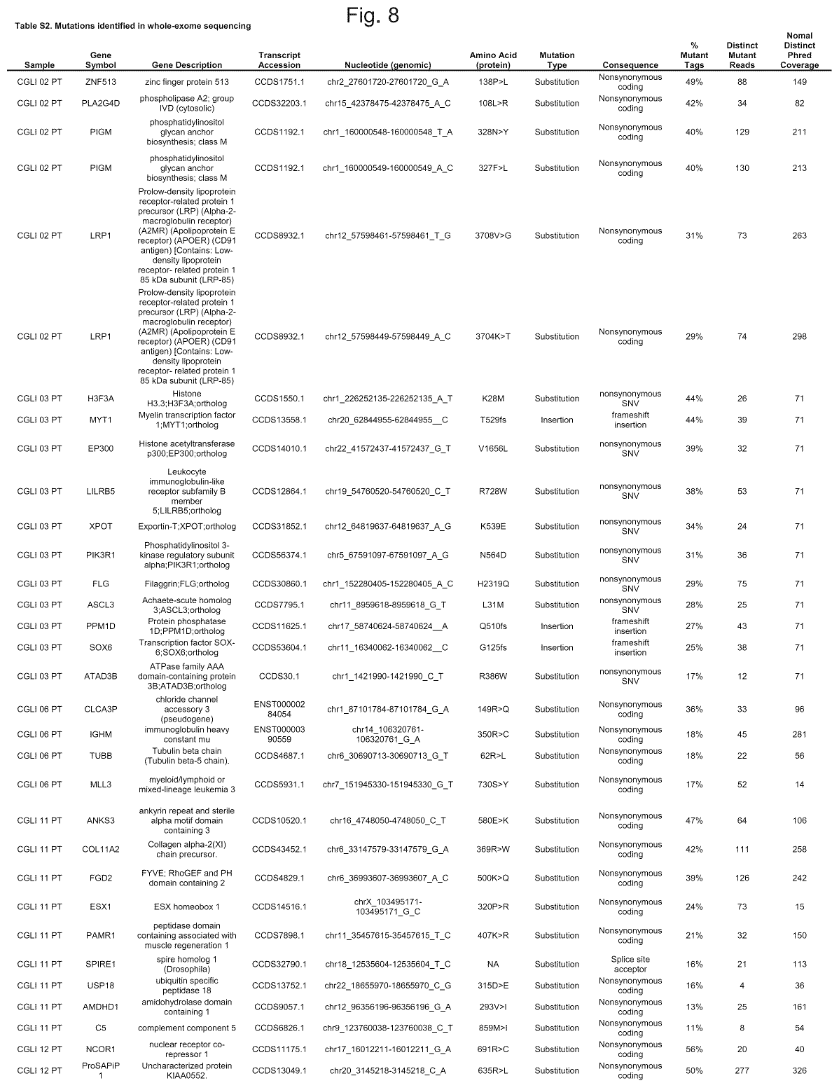

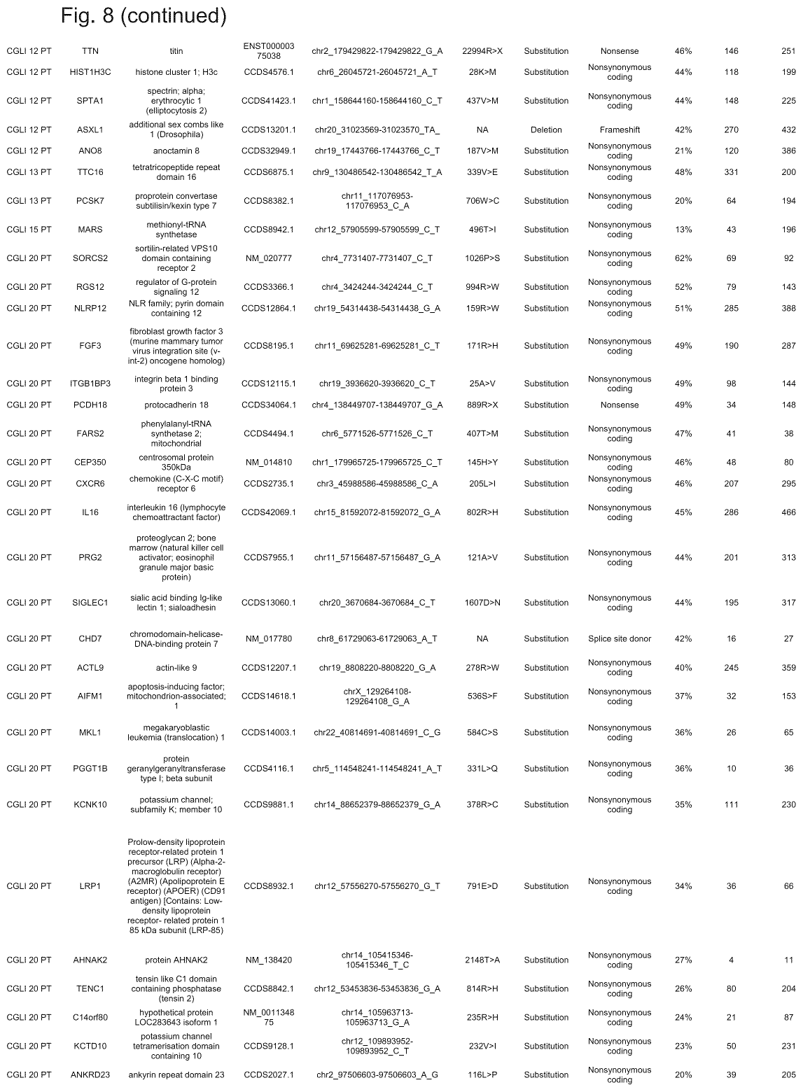

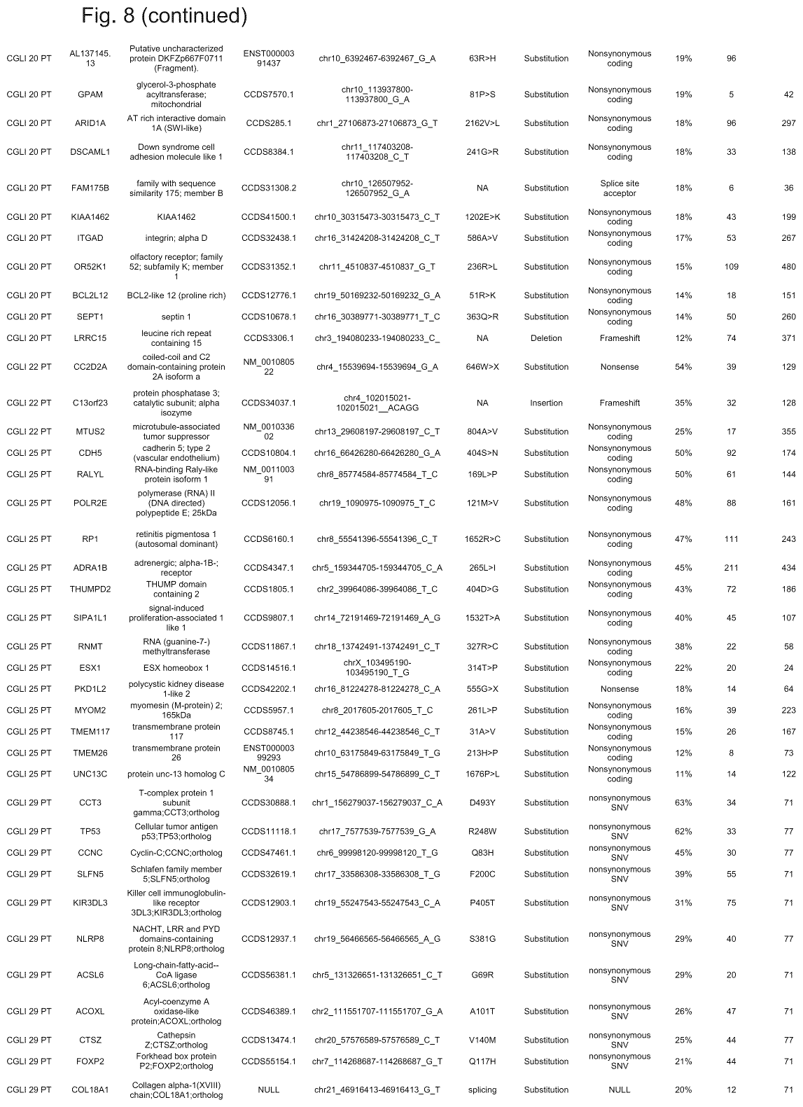

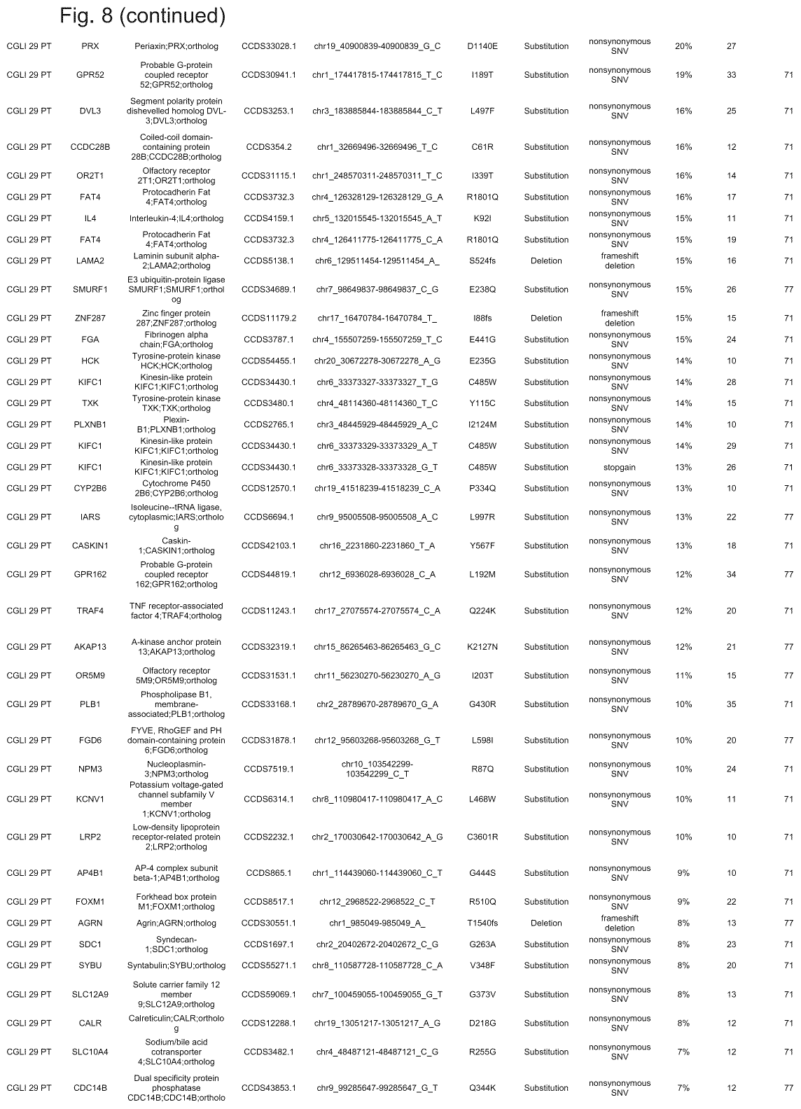

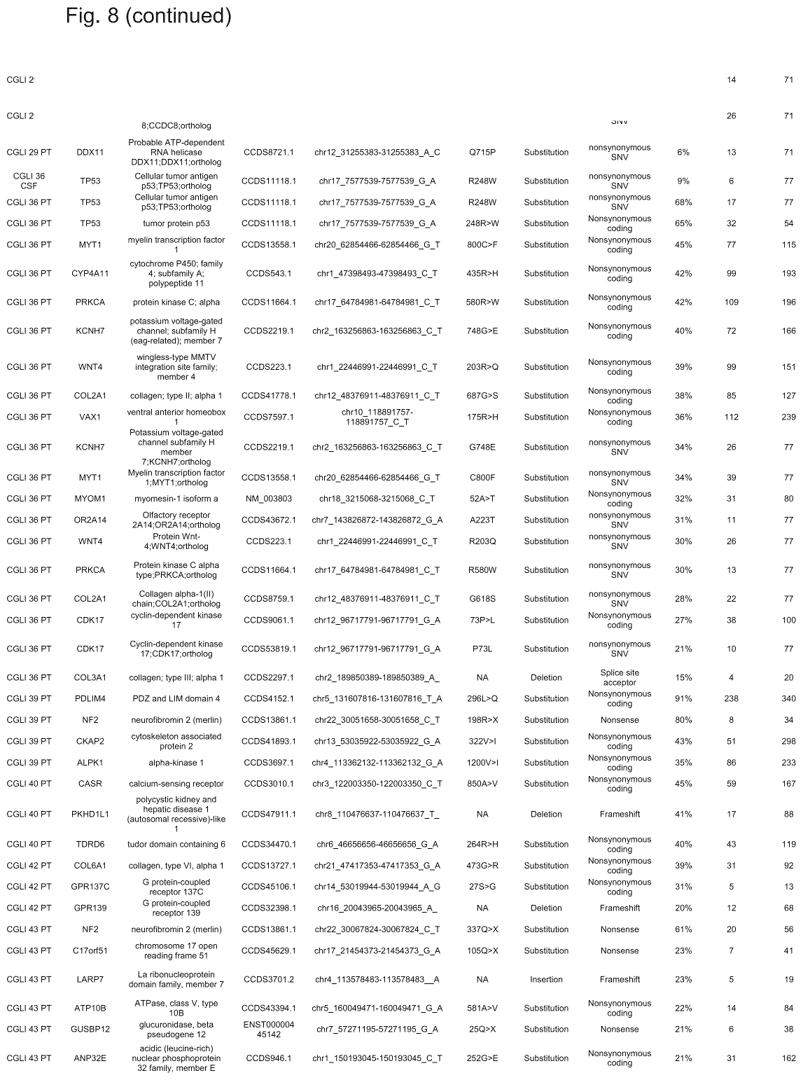

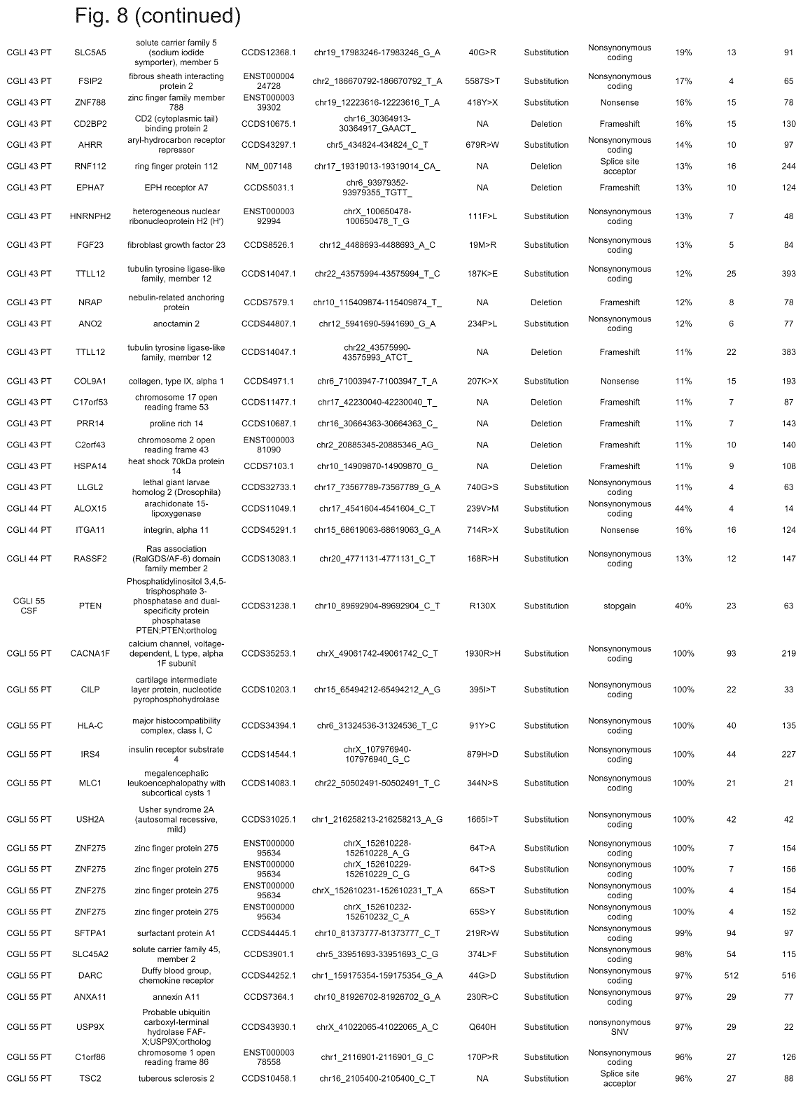

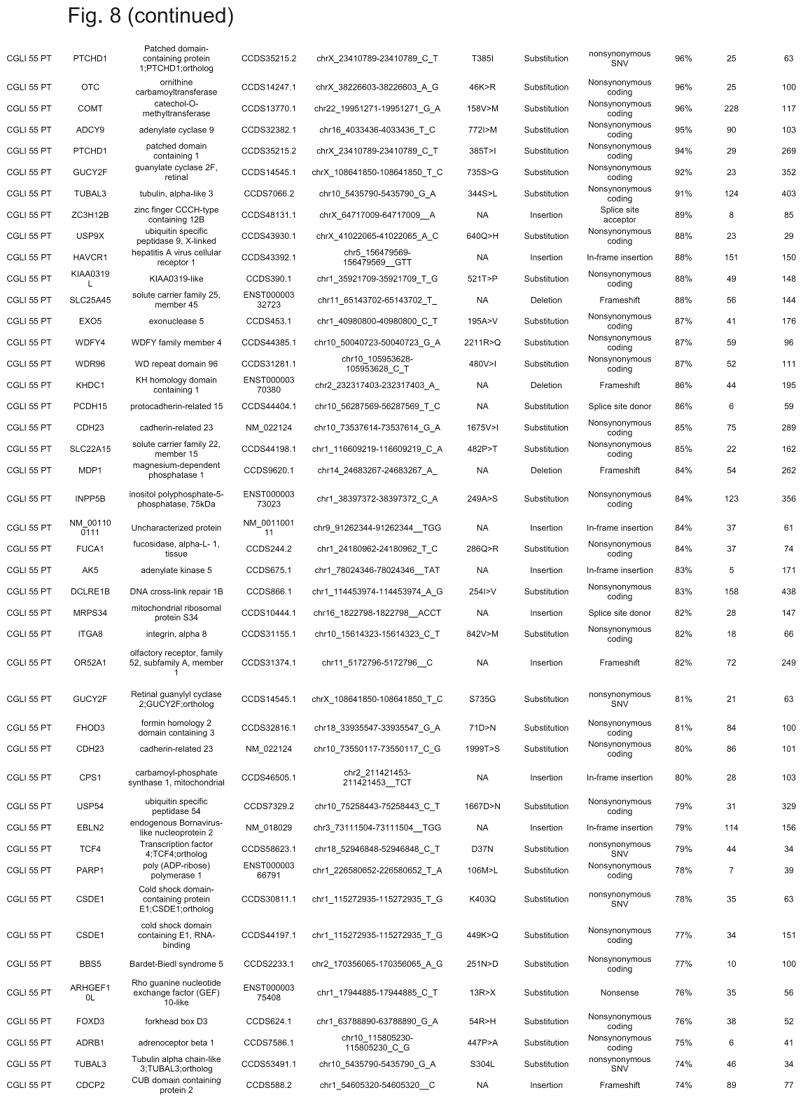

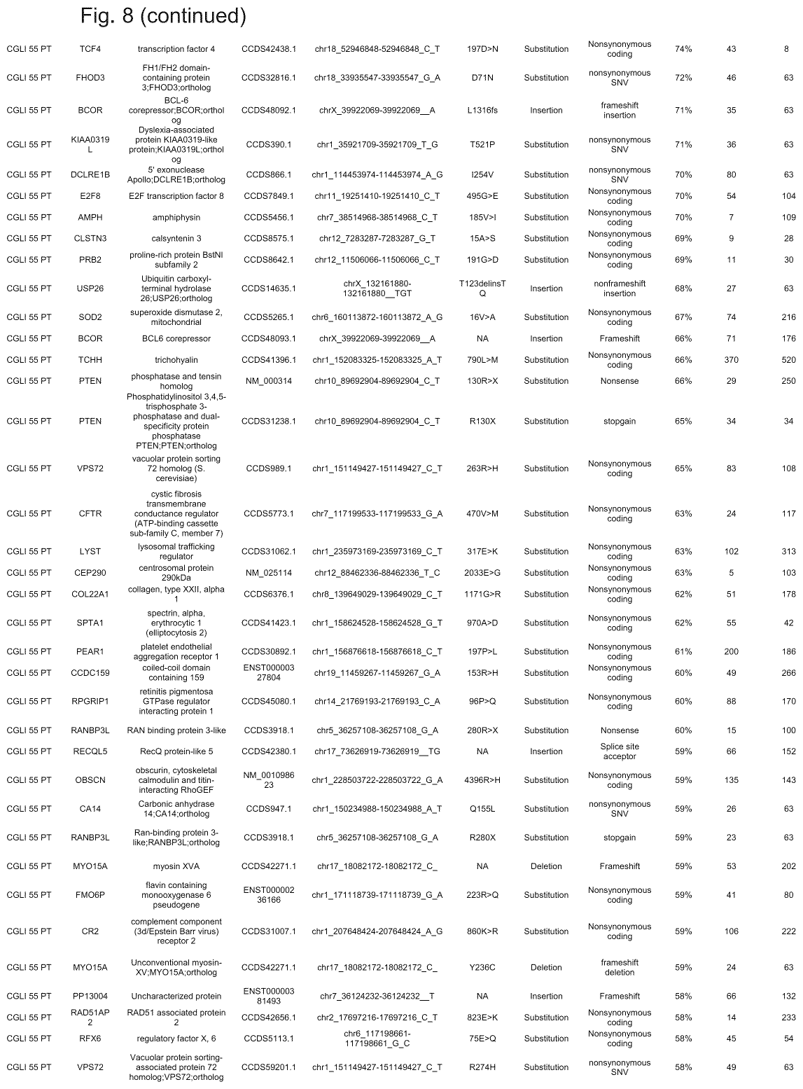

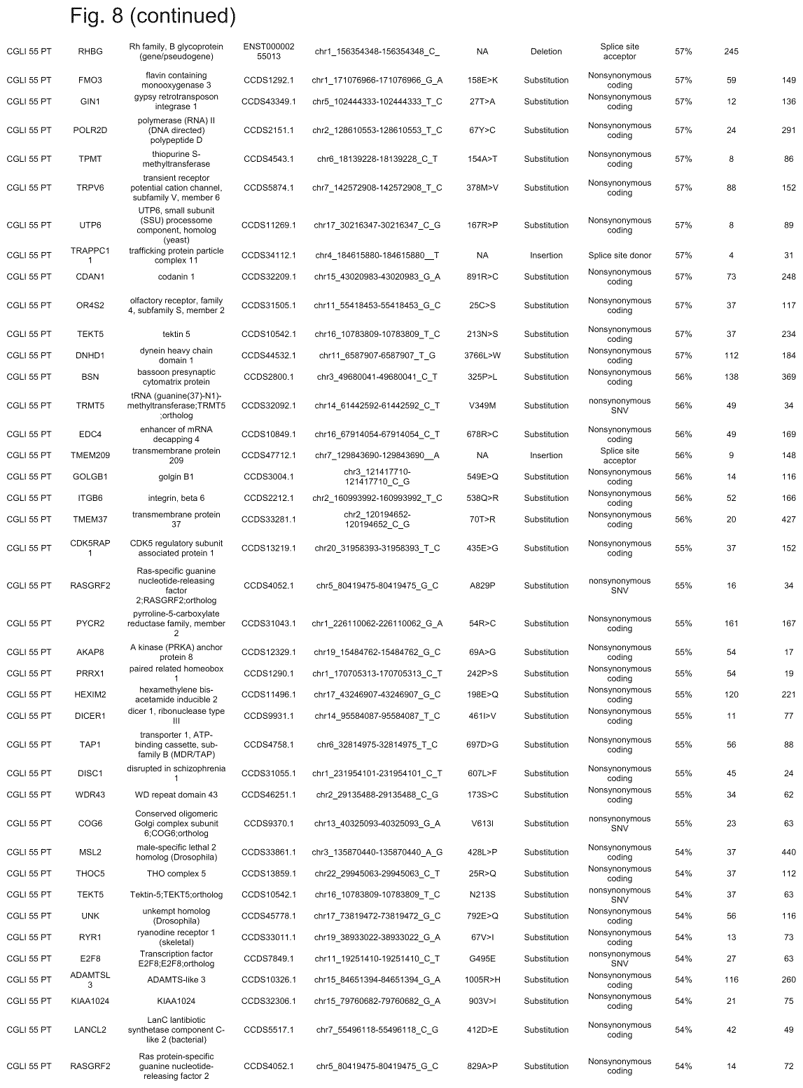

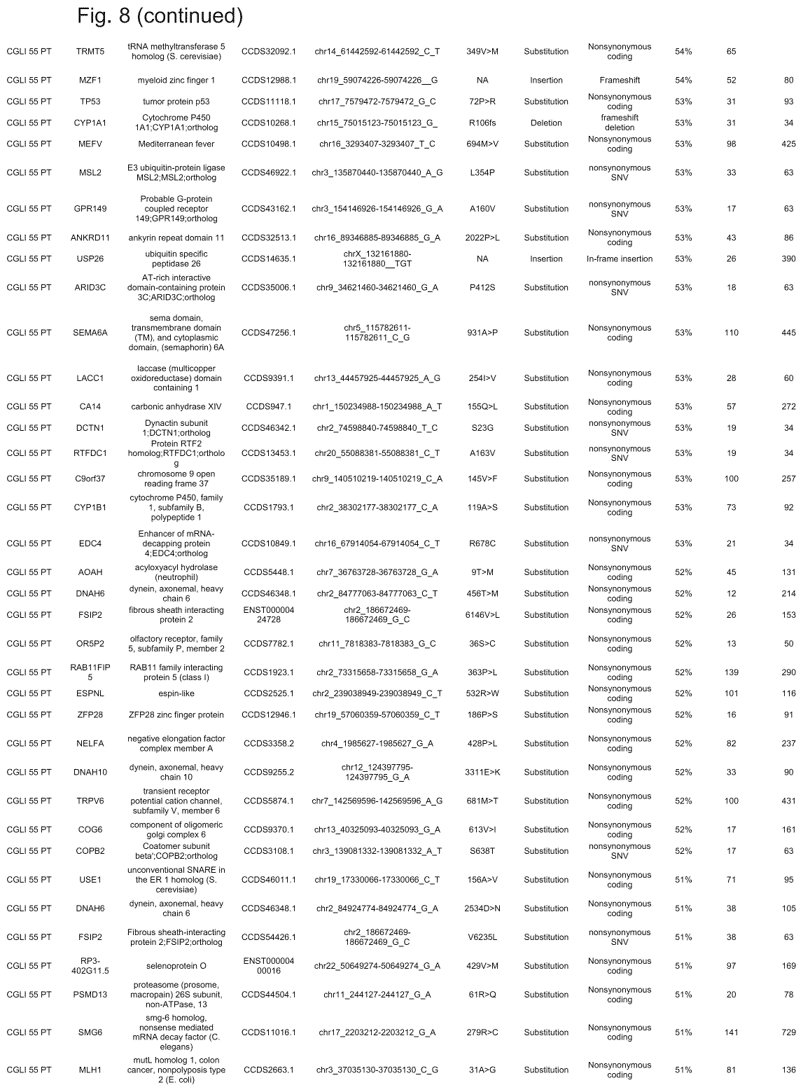

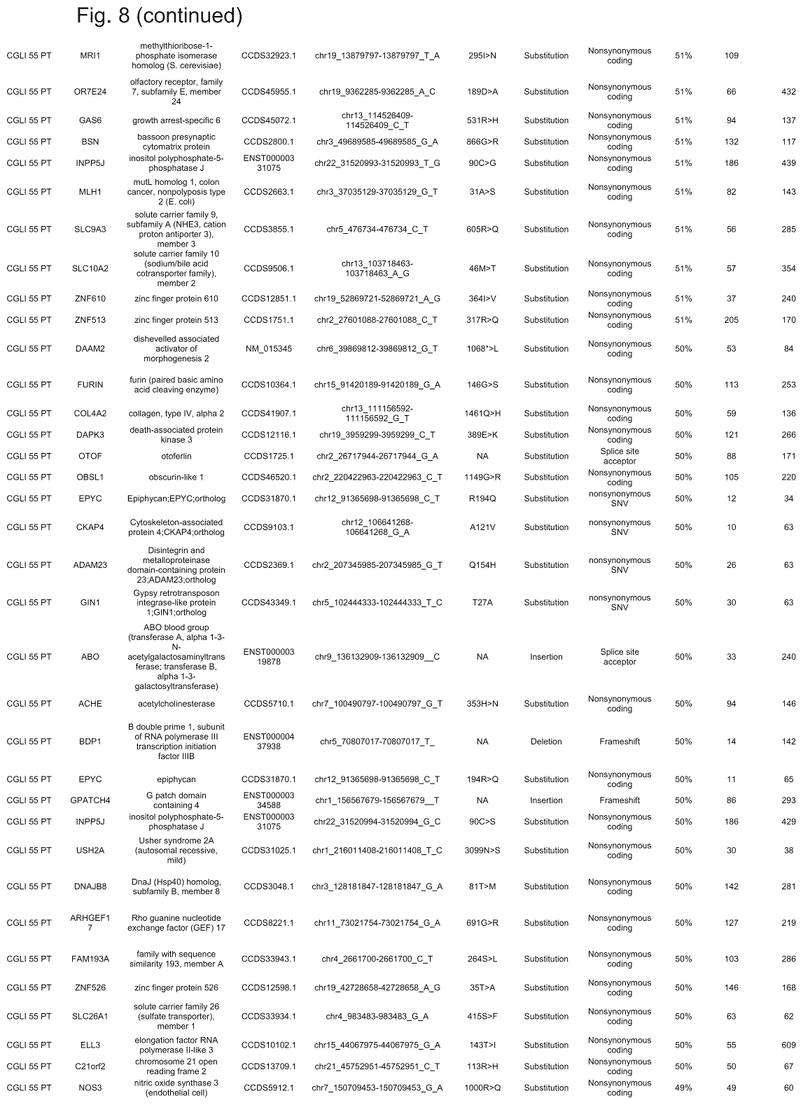

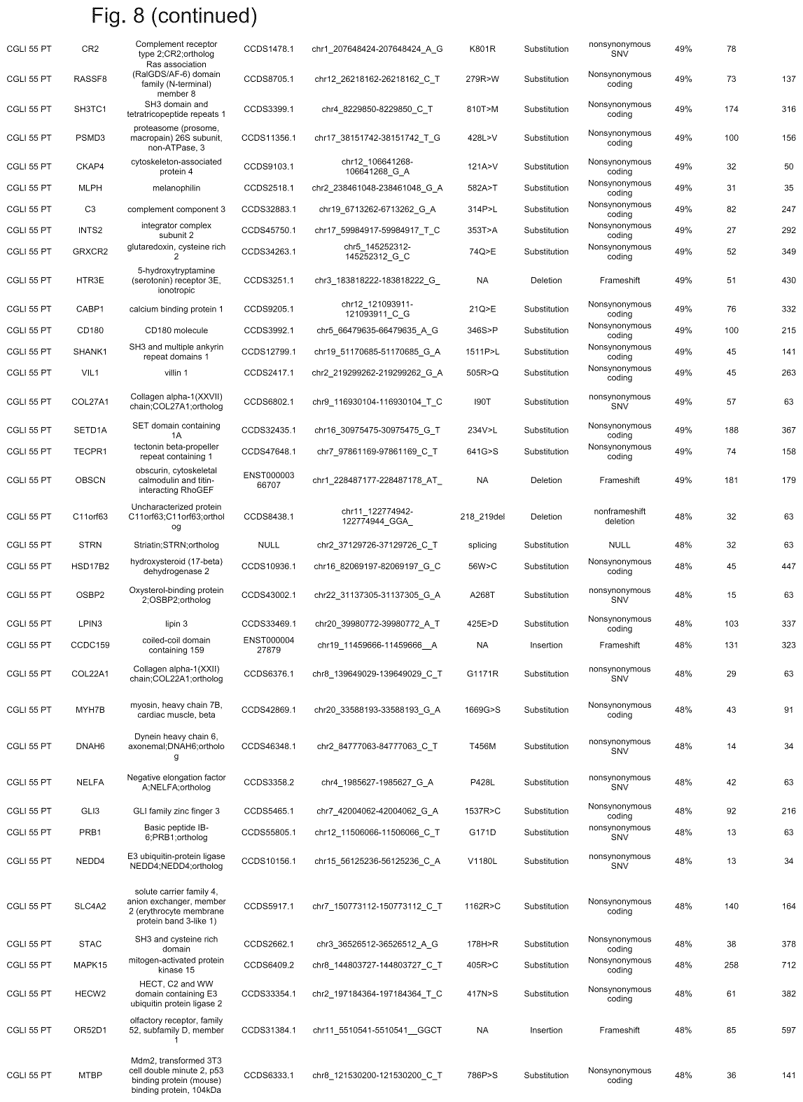

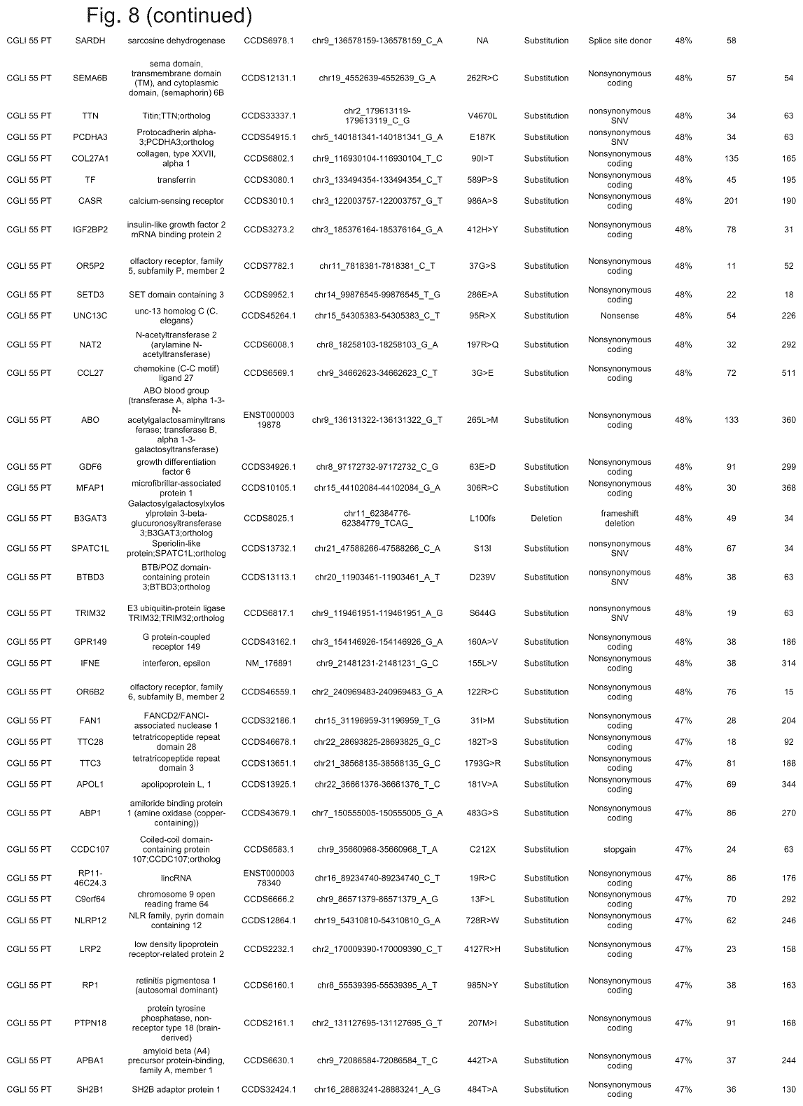

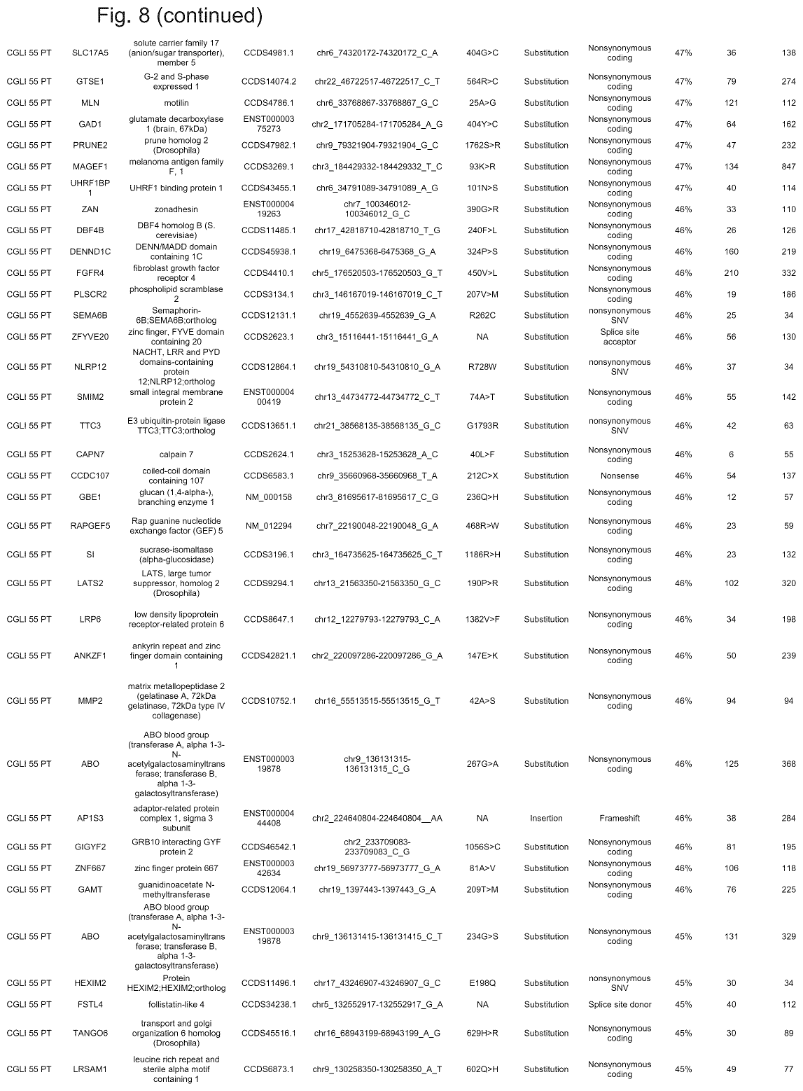

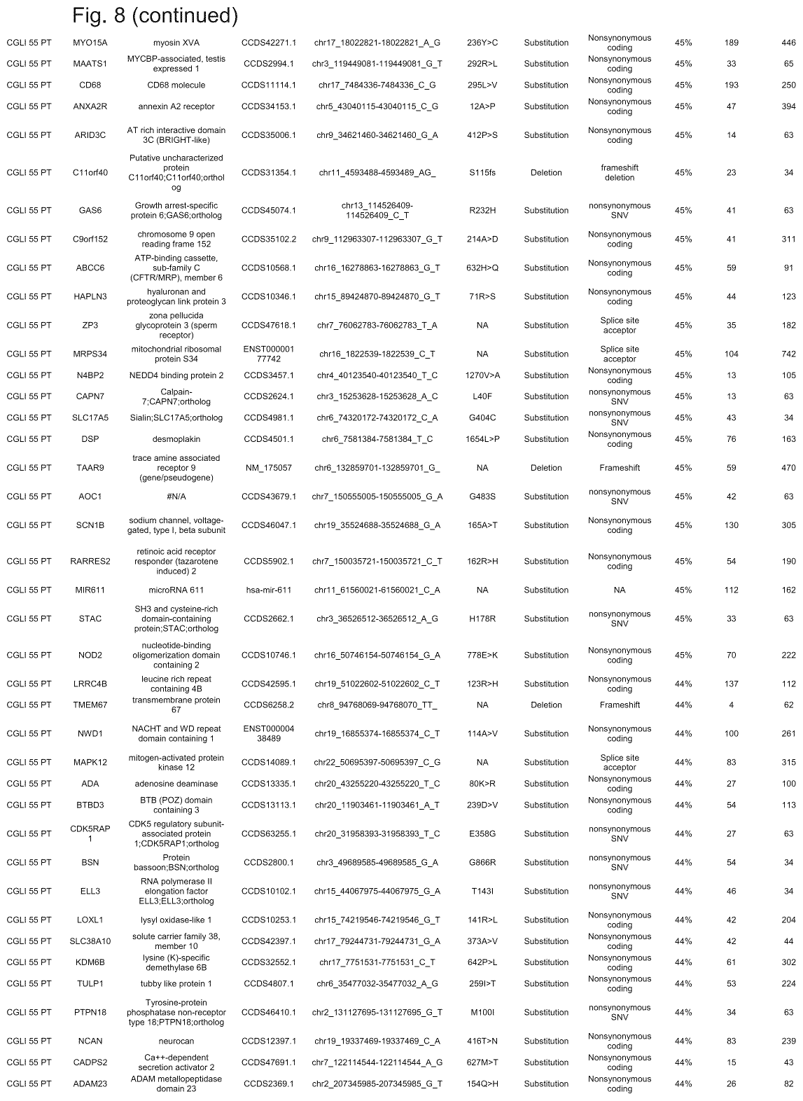

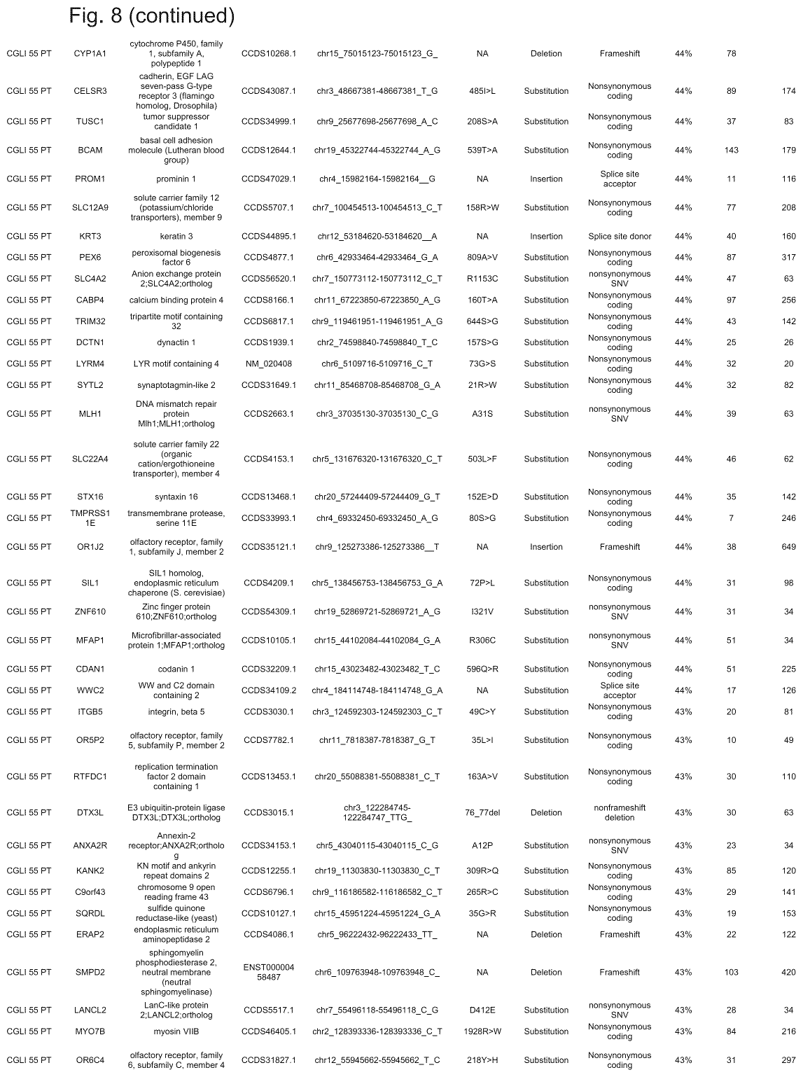

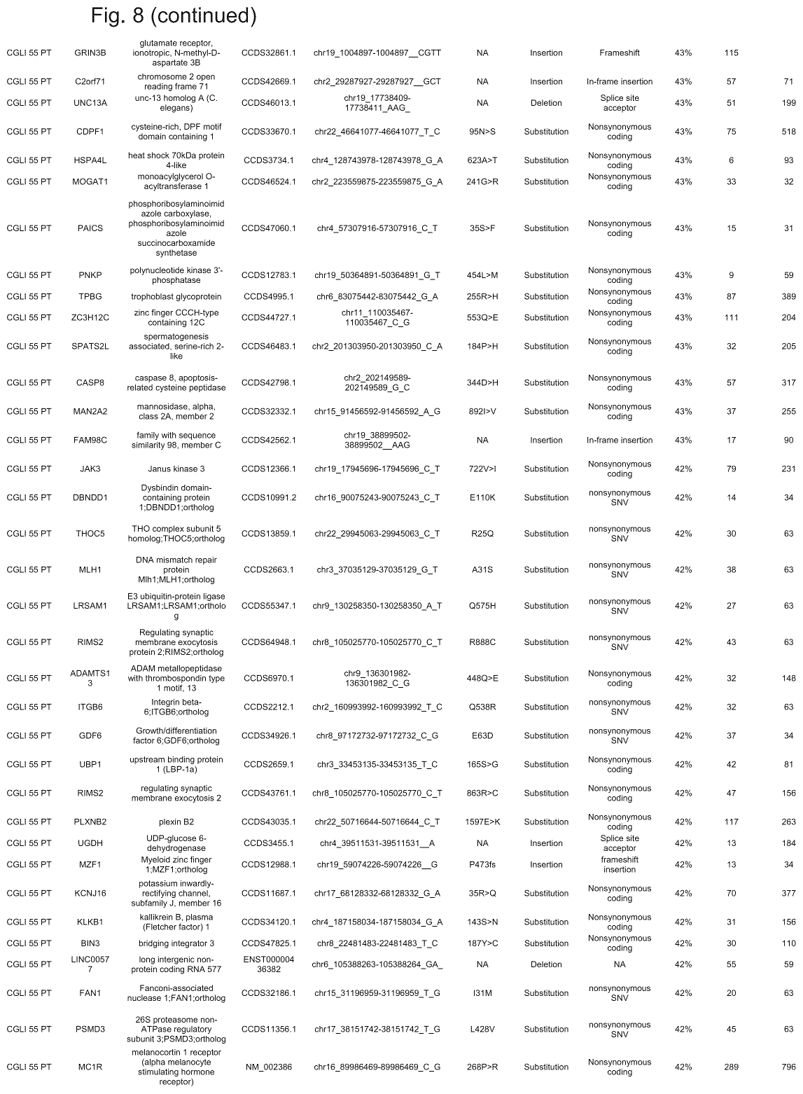

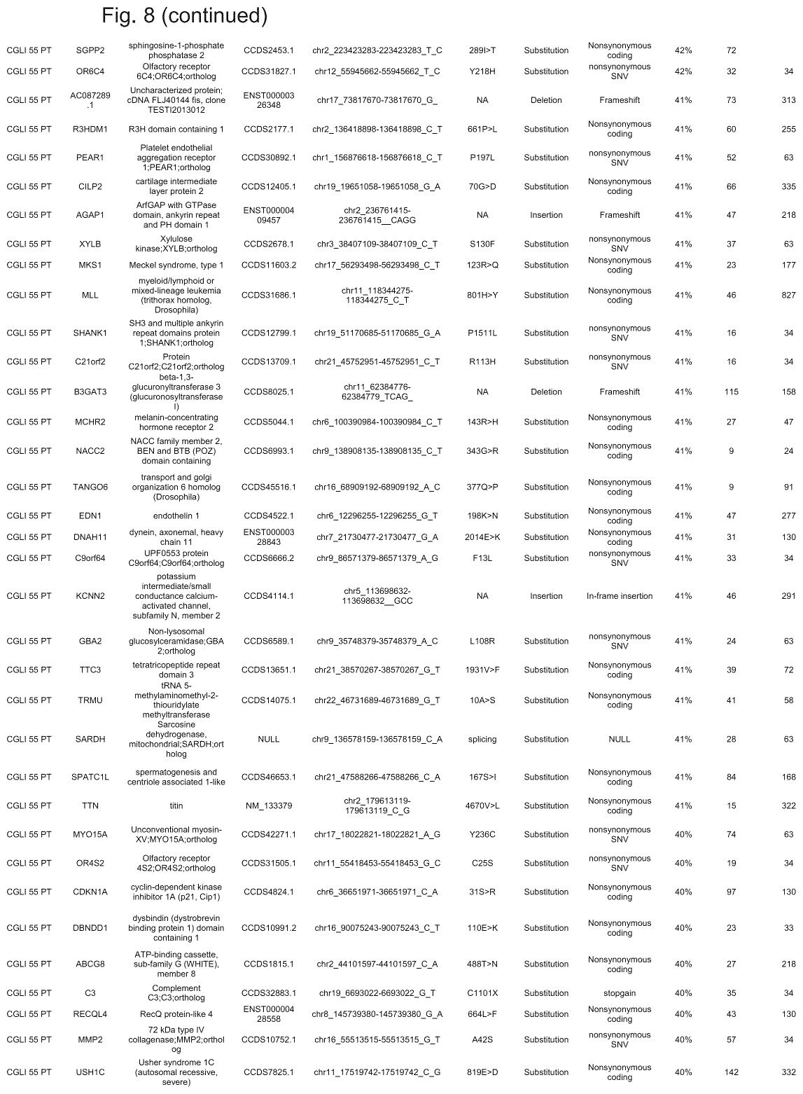

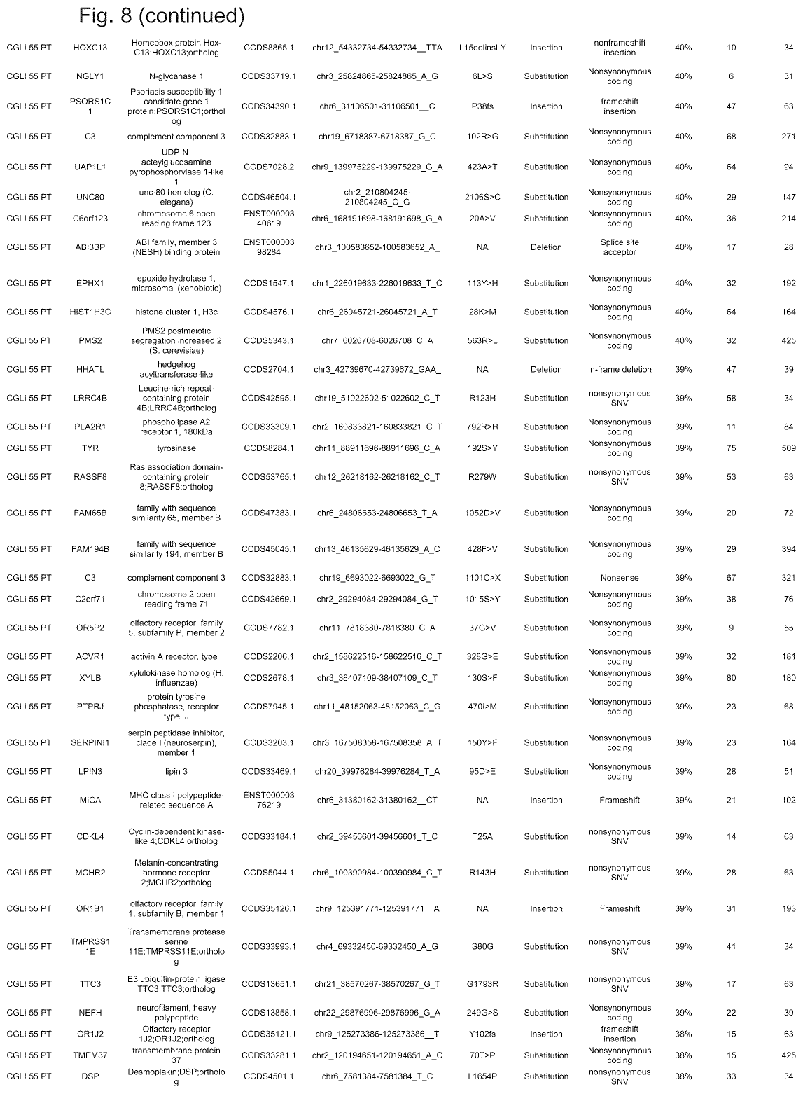

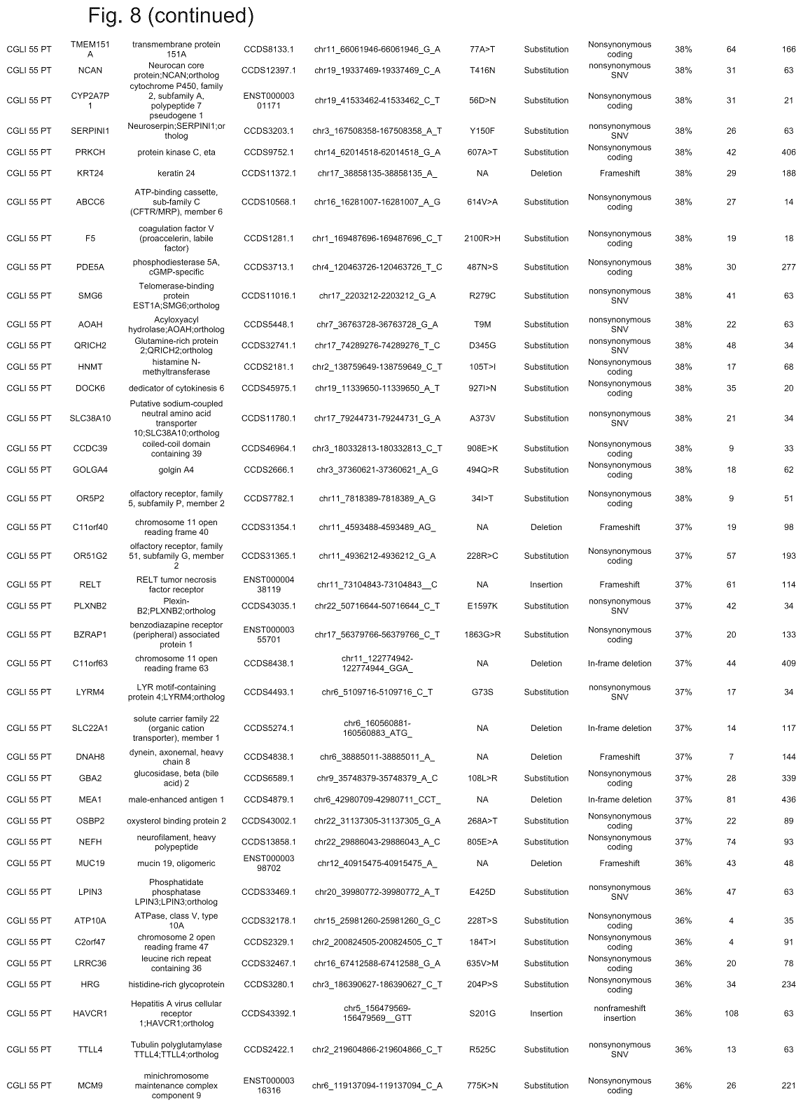

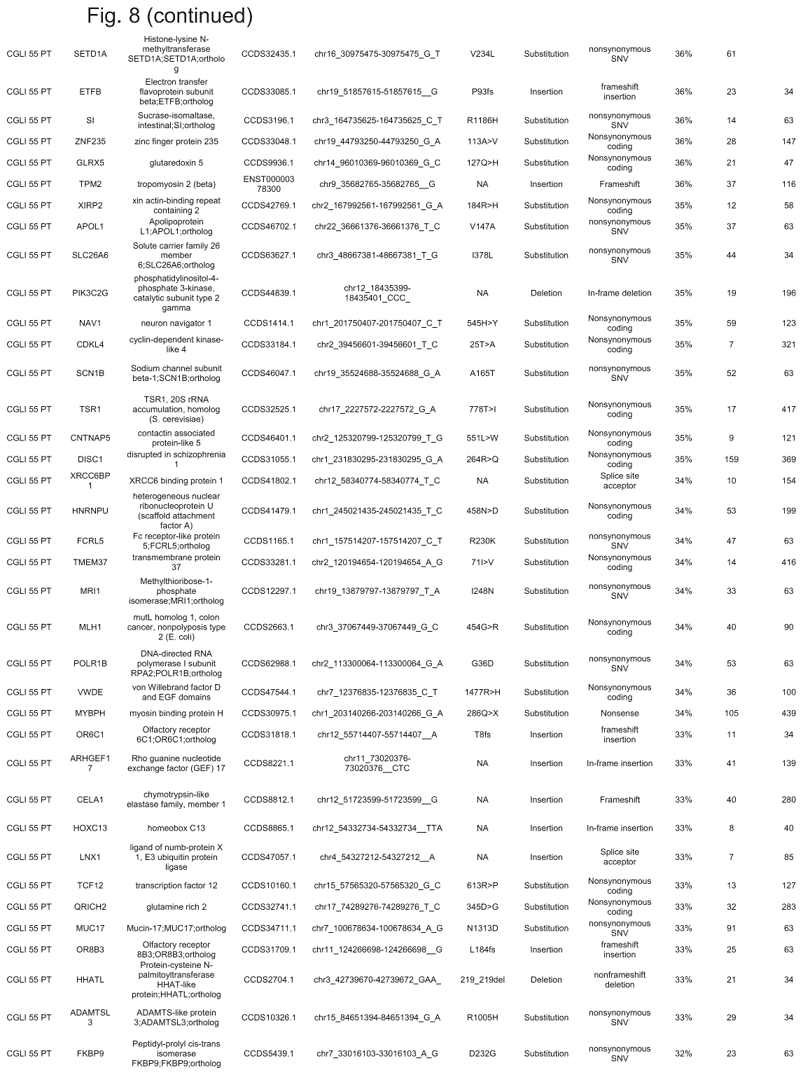

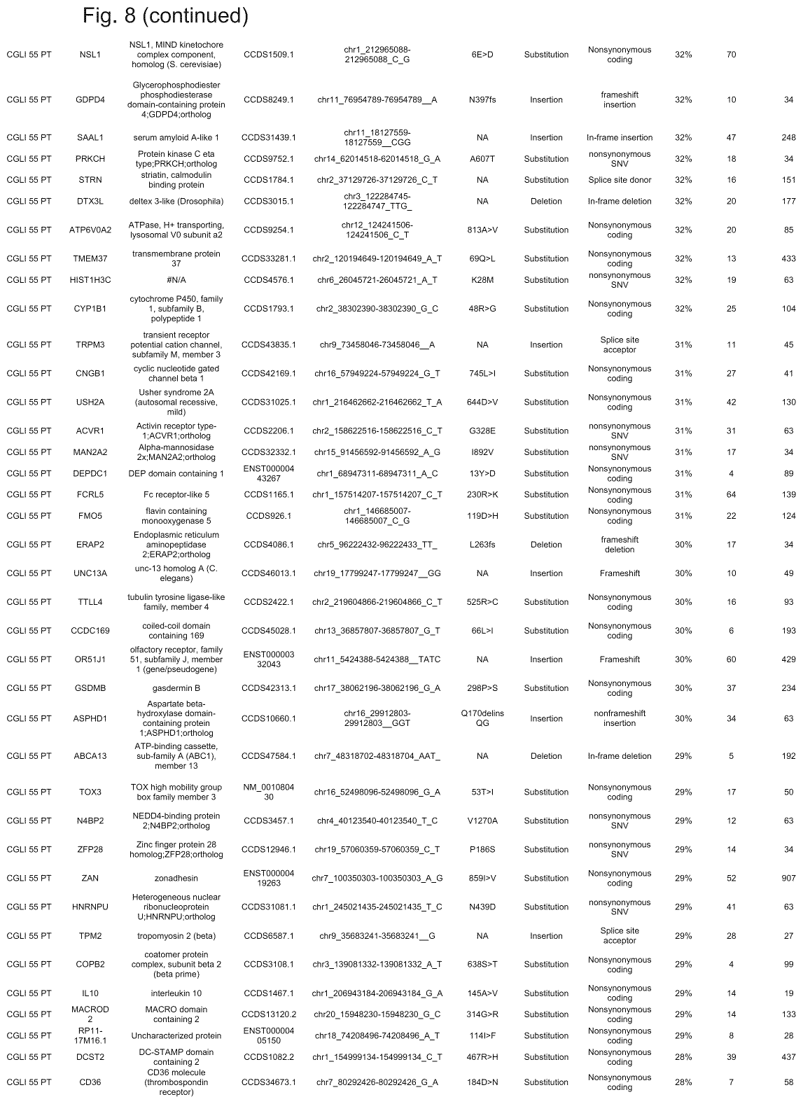

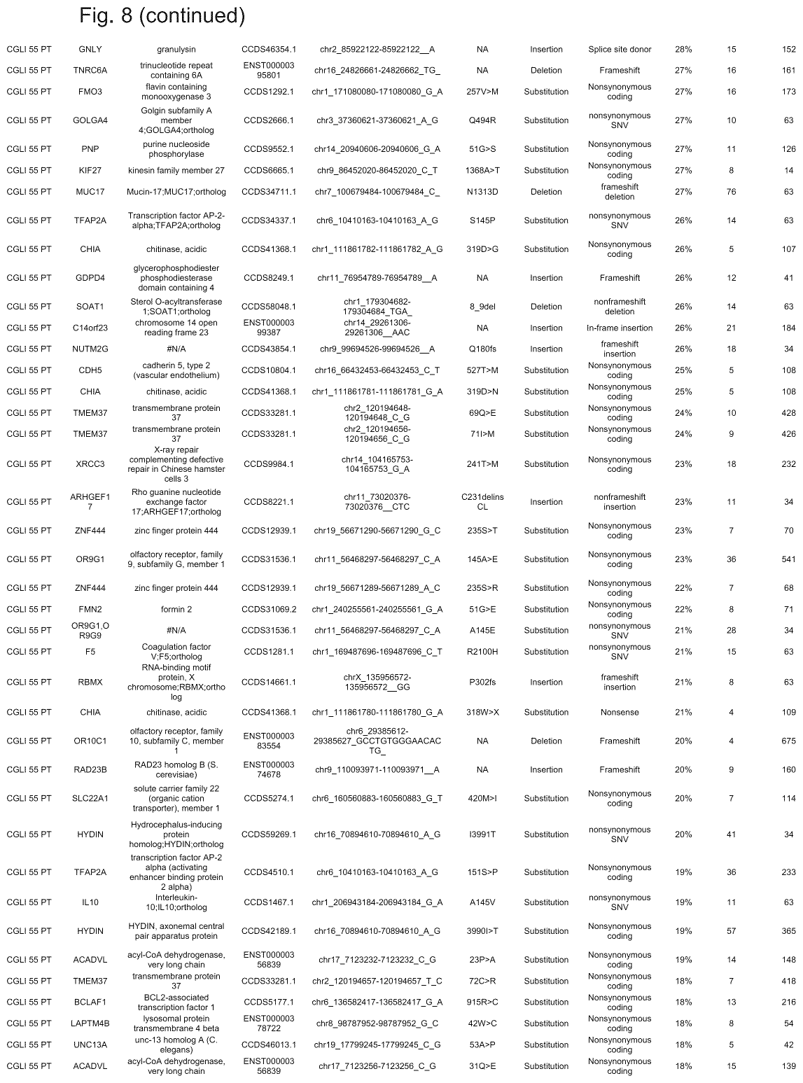

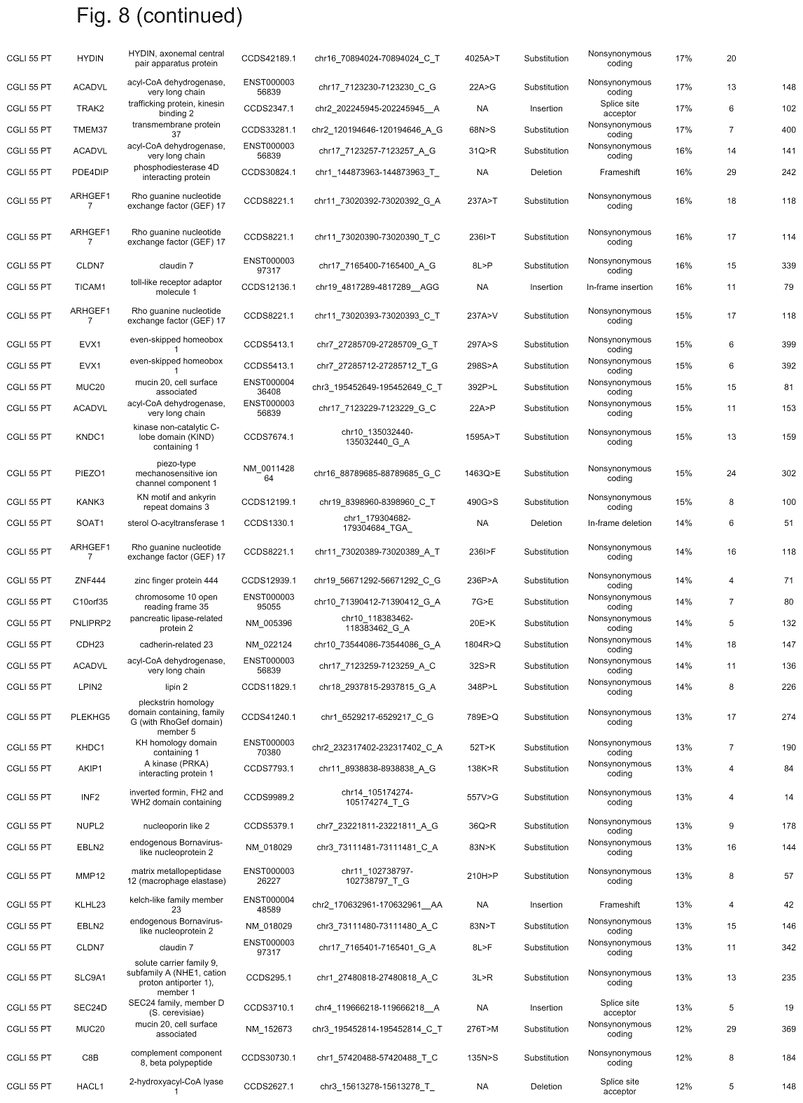

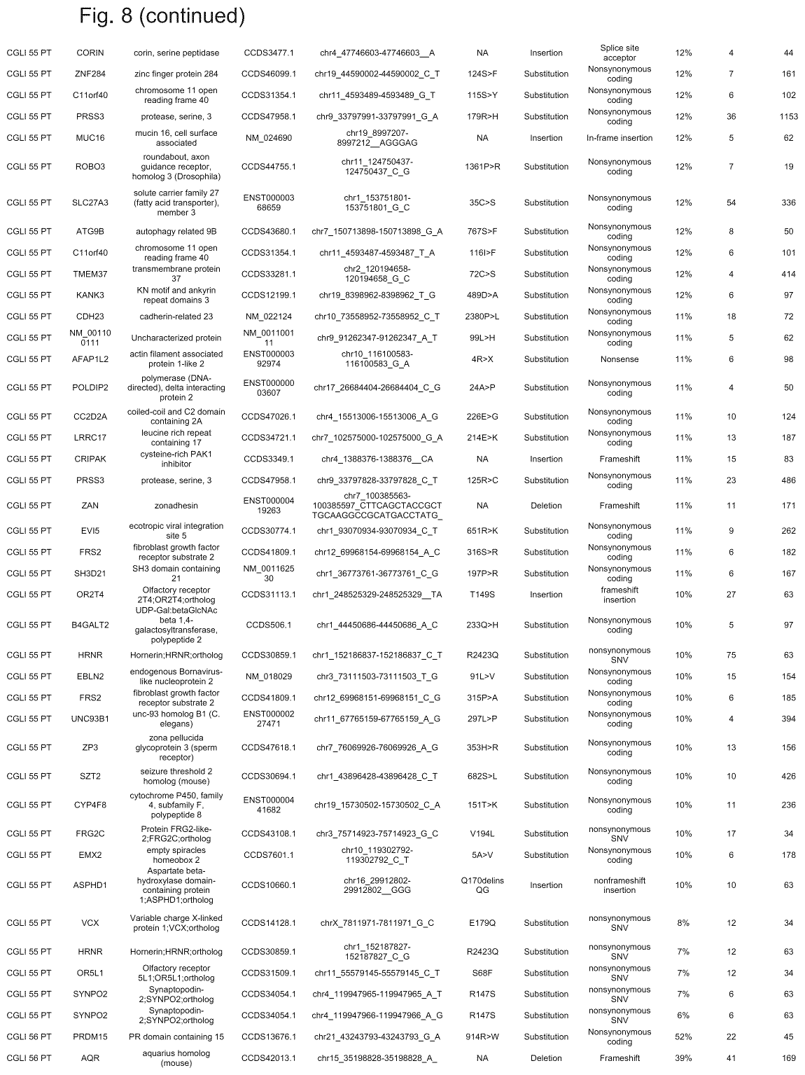

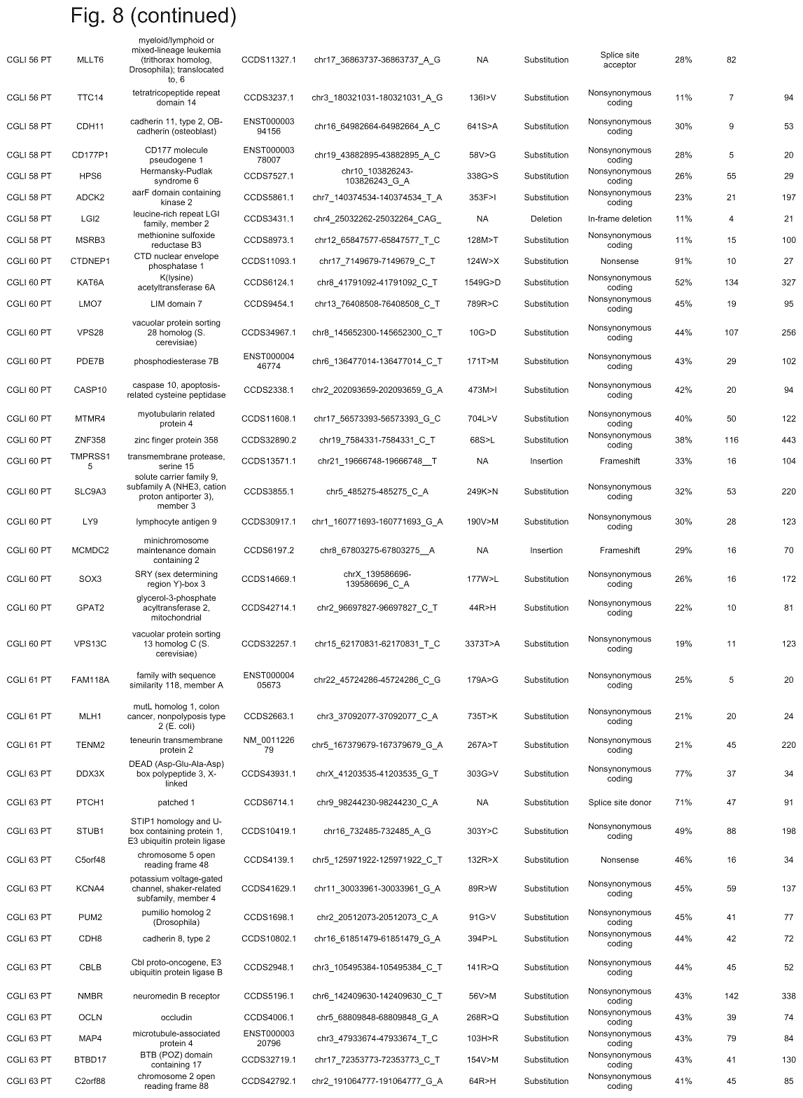

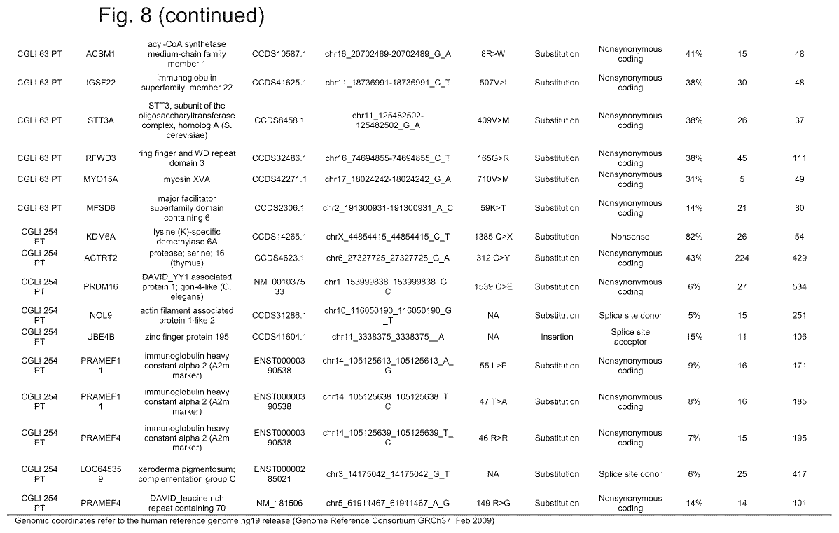

A sequence listing is included as part of this application.

### DETAILED DESCRIPTION OF THE INVENTION

The inventors have developed a way to avoid invasive surgical procedures to determine disease status and/or to avoid treatment delays when radiographic testing fails to show disease progression. Detection of tumor-specific DNA shed from primary CNS tumors into the surrounding cerebrospinal fluid (CSF-tDNA), can serve as a sensitive and exquisitely specific marker for quantifying tumor burden without invasive biopsies.

CNS tumors which can be assessed include those of the brain and spinal cord. As discussed below, those tumors which are located close to a ventricle or cortical surface are preferred. Suitable tumors for assessment include without limitation primary and malignant tumors of the brain and spinal cord, including pilocytic astrocytoma, diffuse astrocytoma/low-grade astrocytoma, anaplastic astrocytoma, glioblastomas (also called glioblastoma multiforme, gbm, or grade iv astrocytoma), brain stem gliomas, ependymomas, oligodendrogliomas, mixed gliomas, meningiomas, pituitary tumors, craniopharyngiomas, germ cell tumors, pineal region tumors, medulloblastomas, and primary CNS lymphomas.

Cerebral spinal fluid can be accessed by any practical means. It can be collected during surgery. It can be collected from an intracranial space, e.g., a space created during and remaining after sugery. It can be collected via lumbar puncture or cisternal puncture. It can be collected using an implanted reservoir, such as an Ommaya reservoir.

While mutations that are detected in the CSF may be previously tested and confirmed in tumor tissue and normal tissue, this may not always be necessary. For example, certain mutations by their prevalence and or location may be presumed to be mutations. In some cases it may be preferred to identify a mutation in tumor tissue prior to assessing it in CSF. In other cases direct detection may be used. When a mutation has been identified in tumor tissue in advance, more targeted assessments can be used, for example, using mutation specific or mutation targeted probes or primers. When a mutation has not been previously identified in tumor tissue, broader screening techniques can be used.

Targeted reagents for assessment of particular mutations will typically be probes or primers. These nucleic acid reagents may optionally contain tags, including unique tags. These nucleic acid reagents may optionally contain labels, such as radionuclides, chromophores, binding sites for binding partners. The reagents may contain nucleic acid modifications that render the reagents more stable, for example. The probes and primers will typically be between 10 and 100 bases, and may be supplied as either double stranded or single stranded molecules. The probes and primers may hybridize to either the strand of the genomic DNA, coding or non-coding. Typically primers will hybridize to opposite strands of the genomic DNA. Desirably the probes and primers will be within 1 kbp, within 500 bp, within 250 bp, within 200 bp, within 100 bp, or within 50 bp of the mutation which is being assessed. Primers can be used in amplification reactions of various sorts. Optionally, amplification can be done using nested primers.

In order to ascertain whether the tumor is located near a ventricle or cortical surface, any type of imaging known in the art can be used. These include without limitation, magnetic resonance imaging, computed tomography, X-rays, positron emission tomography

(PET), functional magnetic resonance imaging (fMRI), and angiography.

In many cases surgery will be the first or one of the first treatments for a CNS tumor. The assessment of CSF in those cases will typically be performed after surgery. However, it may be desirable to assess the CSF even before surgery. In cases where surgery is not indicated, CSF can be assessed directly. The CSF may be assessed at multiple time points during or following a therapy.

Assessment of CSF nucleic acids may be useful in many different contexts. It can be useful for monitoring disease burden. It may be useful for distinguishing neoplastic from non-neoplastic processes. It can be useful for follow-up to a radiological finding.

Minimally invasive techniques to monitor disease burden have been a challenge for nearly all diseases of the CNS, including cancer. This challenge is highlighted by the high risks associated with neurosurgical procedures and the widely recognized limitations of current imaging modalities. In cancer patients, there is no reliable way of parsing out treatment effects from tumor recurrence, causing many patients to undergo unnecessary repeat surgeries. For example, in approximately 30% of patients with glioblastoma who undergo a repeat resection for presumptive recurrence, pathologic examination of the resected specimen reveals necrosis, scarring or other treatment-related effects rather than recurrent disease (20). Conversely, while patients are waiting for or recovering from surgery, chemotherapy or radiation therapy cannot be administered, providing time for unabated tumor growth. Finally, patients are often kept on ineffective medication regimens until definitive signs of tumor progression appear on imaging. This delay in detection precludes potential opportunities to undergo new targeted therapies that might be effective for their disease (21). The health costs of these missed opportunities will increase with the expected advances in therapeutic modalities.

Given the need for sensitive and specific markers to monitor tumor dynamics, we asked whether tumor-derived DNA could be found in the cerebrospinal fluid of patients with primary CNS tumors. This study was stimulated by our inability to frequently detect tumor DNA in the plasma of these patients (8) and inspired by previous demonstrations that tumor-derived DNA could be found in fluids located in the proximity of neoplastic lesions. For example, a recent pilot study by Pan et al suggests that tumor-derived DNA can be detected in the spinal fluid of individuals whose primary tumors have metastasized to the brain (22). Though lumbar puncture to obtain CSF is not a non-invasive procedure, it qualifies as minimally invasive and is currently routinely performed to follow some brain tumor patients, particularly those with medulloblastomas (23, 24). Unfortunately, the examination of these CSF samples by cytology is usually of limited utility (25, 26). Only one of the 35 patients evaluated in the current study had concomitant cytologic studies of CSF, precluding direct comparison. The results of this study suggest that the rates of tumor-derived DNA found in the CSF (74%) closely approximate the levels found in body fluids adjacent to other tumor types. For example, urine in bladder cancer was found to have tumor derived DNA in 70% of cases, while sputum in lung cancer was positive in 79% of cases (27, 28). While the rate of detection observed in the current study was not 100%, its sensitivity was comparable or superior to other non-invasive tests for malignancies in general. Moreover, and as noted below, it was particularly sensitive for tumors that abutted a CSF reservoir or cortical surface. Finally, from a technological standpoint, the average fraction of mutant DNA (12.2%) far exceeded the limit of detection of the sequencing assay used (0.01%). This assay could be performed with any commercially available next-generation sequencing instrument, at relatively small cost.

Our study revealed a significant association between the location and type of the tumor with the presence of CSF-tDNA. In particular, we were able to detect all thirteen WHO Grade III or Grade IV gliomas (also known as anaplastic astrocytoma and glioblastoma, respectively), all five medulloblastomas and all three ependymomas that abutted a CSF reservoir or cortical surface. It is in these aggressive tumors where the need for a robust biomarker is most desperately needed. There are also emerging data that some brain tumors, particularly those with genotypes susceptible to targeted therapies, may be able to be treated primarily with medical therapies, thereby obviating the need for surgery if appropriate non-invasive diagnostic tools were available (29-32). It is also worth noting that surgical resection nearly always creates an opening extending from the surface to the deep-seated tumor. This passageway typically persists and may enable tumor-derived DNA from any residual or recurrent tumor to enter the CSF. Even without such surgically-induced openings, the vast majority of medulloblastomas and ependymomas arise within or communicate with a ventricular reservoir, making them well-suited for CSF monitoring. Indeed, these are currently the most common tumor types in which CSF cytology is a part of routine clinical practice (24, 33). Future studies will be required to directly compare CSF-tDNA and CSF cytology. However, despite being the standard of care, CSF cytology has reported sensitivities below 50% and often requires a large volume of CSF for analysis (>10 ml) (34). Rather than replacing cytology, we envision that CSF-tDNA will be used in combination with it and other biomarkers under development, as well as with radiographic and clinical parameters, to substantially increase the accuracy of tumor burden estimates post-surgically (35-38).

Given the invasive and risky nature of surgical interventions on the brain and spinal cord, it would be useful to be able to identify a neoplastic process without performing surgery. Our results provide a glimpse of the potential for this form of diagnosis in the future. We evaluated four patients, one with a tumor in the midbrain, one in the pons, and two in the spinal cord. Using WES, we were able to detect CSF-tDNA in two of the four cases by comparing the data to that obtained by targeted sequencing with SafeSeqS. The results were consistent with expectation in that the mutant fractions revealed by genome-wide sequencing were in accord with those identified by targeted sequencing (Table 4). Additional cases will need to be tested in order to elucidate the potential of this approach in patients in whom biopsies are challenging, but our results show that genome-wide analysis of the DNA from CSF is feasible in at least some cases.

While the results described above are promising, we caution that this is an exploratory study designed primarily to determine whether it was possible to detect CSF-tDNA in patients with primary CNS tumors. A secondary goal was to document the anatomical and pathologic characteristics of the tumors that shed DNA into the CSF. The most important technical limitation of our study is that CSF samples were obtained at the time of surgery, often from the ventricles, rather than from a lumbar puncture. CSF has been demonstrated to quickly circulate throughout the ventricles and spinal reservoirs (39, 40), It is therefore very likely that the DNA in the spinal fluid obtained via lumbar puncture will be similar to that of the ventricles, even though the fluid obtained from lumbar puncture is farther away from the site of malignancy. An additional consideration is that in individuals with a bulky mass that obstructs spinal fluid flow or elevates intracranial pressure, a lumbar puncture might be unsafe. However, these patients will almost always require surgical decompression to reduce the mass effect generated by the tumor and CSF could be safely obtained after opening the dura. The exact method and location of CSF sampling in patients with CNS neoplasms will need to be individualized and will be based on a number of factors, including tumor location, case of CSF sampling and clinical characteristics. For example, patients may initially undergo CSF sampling from an intracranial space at the time of surgery to determine baseline levels of CSF-tDNA but lumbar punctures could be used to longitudinally monitor CSF-tDNA levels.

Now that it has been documented that most primary brain tumors release tumor DNA into the CSF, the stage is set for a longitudinal study of the clinical utility of this new biomarker. Our results suggest specific guidelines for such a follow-up study. The optimal patients to follow would be those with medulloblastomas, ependymomas, or high-grade gliomas that abut a CSF space, as the CSF-tDNA assay is particularly sensitive in such cases and these tumor types are relatively common. CSF-tDNA should be evaluated intra-operatively to establish a baseline, and a concomitant lumbar puncture should be performed when possible to ensure concordance between the two fluid samples. Subsequent evaluations of CSF obtained through lumbar puncture or an implanted reservoir should be compared to other clinical and laboratory features, with the goal of determining the utility of CSF-tDNA to detect minimal residual disease. For example, patients whose mass persists upon MRI but whose CSF-tDNA is undetectable might be spared a second biopsy. Alternatively, patients in whom residual disease is evident upon CSF-tDNA analysis, but is equivocal upon imaging analysis, might be well-served by additional therapy. In the future, it is likely that most brain tumors will be routinely assessed for mutations in various genes of interest, both for prognostic and therapeutic purposes (41-43). The availability of such sequencing data should make the approach described here more cost-effective and easier to implement.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLES

We analyzed 35 primary CNS malignancies and found at least one mutation in each tumor using targeted or genome-wide sequencing. Using these patient-specific mutations as biomarkers, we identified detectable levels of CSF-tDNA in 74% (95% CI 57 to 88%) of cases. All medulloblasomas, ependymomas, and high-grade gliomas that abutted a CSF space were detectable (100% of 21 cases, 95% CI 88% to 100%), while no CSF-tDNA was detected in patients whose tumors were not directly adjacent to a CSF reservoir (p<0.0001, Fisher's exact test).

### Example 1

### Materials and Methods

Patient Samples. All samples were collected after approval was obtained from the Johns Hopkins Institutional Review Board and informed consent was provided. Whole blood and cerebrospinal fluid were collected at the time of surgery, prior to surgical manipulation of the tumor. A white blood cell (WBC) pellet was prepared from the blood sample after hypotonic lysis of red blood cells via centrifugation at 200 g. CSF was frozen in its entirety at −80° C. until DNA purification, and the entire volume of CSF (cells plus fluid) was used for DNA purification. The amount of CSF used averaged 4.8 ml (range 0.75 to 10 mL). When fresh tumor tissue from surgical specimen was available, it was immediately frozen at −80° C. When frozen tissue was not available, formalin-fixed, paraffin-embedded (FFPE) tissues were used for DNA purification. In either case (fresh-frozen or FFPE) tumors were macro-dissected to ensure neoplastic cellularity exceeding 50%. DNA was purified from the white cell pellet, CSF and tumor using an ALLPrep kit (Qiagen, cat #80204).

Statistical Analysis. Clinical characteristics were compared between the CSF samples with and without detectable CSF-tDNA with Fisher's exact test or t-test. Correlation coefficients among outcomes were estimated using Pearson correlation statistics. A logistic regression model was used to estimate the odds of detecting CSF-tDNA under different conditions. All p-values are 2-sided and all analyses were conducted using SAS software (version 9.2, SAS Institute).

Tumor Mutational Profiling. A tiered approach was utilized to determine a somatic mutation within each tumor. Initially, a PCR-based approach testing for mutations in codons 130 to 139 of IDH1, codons 126 to 155, 144 to 178, and 250 to 262 of IDH2, all coding exons of TP53, and the TERT promoter was employed (44-48). If no mutations were present within these genes, paired-end libraries of DNA from the tumors and white blood cell pellets were prepared and captured (SureSelect, Agilent) as previous described (47). Massively parallel sequencing was carried out on an Illumina HiSeq instrument, either in the Goldman Sequencing Facility at Johns Hopkins Medical Institutions or at PGDx. Mutations were identified as previously described (47, 49-52).

Mutation Detection in CSF. DNA from tumor, WBCs, and CSF was used to validate the somatic mutations identified by targeted sequencing and determine whether these mutations could be found in the CSF. Three to five nanogram (ng) of tumor and WBC DNA were used for each assay, while all DNA from the CSF (for cases with <20 ng of CSF DNA available) or 20 ng of CSF DNA was used for each assay (Table 2). For this purpose, primers were designed to amplify a ˜100 bp region surrounding each mutation. The two primers had universal sequences at their 5′-ends allowing a second round of PCR to be performed using a second set of primers containing these sequences (19, 47). The sequences of the primers used to assess each mutation are listed in Table S1. The final PCR products (after two rounds of PCR) were purified with AMPure (Beckman) and sequenced using an Illumina MiSeq instrument. The data were analyzed with the SafeSeqS pipeline, allowing mutations occurring as infrequently as 0.01% to be detected and quantified with confidence using the experimental conditions applied (19). In every case, DNA from the normal cells served as a control to ensure that the mutations were the result of errors generated during the DNA purification, amplification, or sequencing processes. Four paired-end libraries for CSF samples were also generated and exome captured (Table 4). Preparation of genomic library was performed using the TruSeq DNA Sample Prep Kit (Illumina) according to the manufacturer's recommendations. Exomic capture (SureSelect, Agilent) and massively parallel sequencing were carried out as described above.

### Example 2

Patient and tumor characteristics. Thirty-five patients with CNS cancers were enrolled in this study. Their age, gender, race, and pre-operative symptoms are listed in Table 1, together with their tumor characteristics. Six patients had medulloblastomas and twenty-nine had gliomas. Seven, nine, two, and seventeen of the tumors were classified as WHO Grades I, II, III, and IV, respectively. Twenty-nine (83%) of the 35 patients provided CSF during the initial surgery, while the remaining six (17%) did so during a repeat resection. The tumors were distributed throughout the brain and spinal cord, with fourteen arising in the posterior fossa (including six medulloblastomas), eight in the supratentorial compartment of the brain, and thirteen in the spinal cord (Table 1).

### Example 3

Identification of somatic mutations. At least one mutation was identified in each of the 35 tumors analyzed using a tiered approach (targeted sequencing followed by whole-exome sequencing) described in the Materials and Methods section.

With the targeted sequencing approach, we identified mutations in thirteen tumors. The mutations in these samples occurred in TP53 (n=5), IDH1 (n=2), and the TERT promoter (n=6) (Table 2). In the remaining 22 tumors, whole-exome sequencing was used to identify at least one mutation per sample. Genes mutated in these samples included well-known drivers, such as NF2, PIK3R1, PTCH1, and PTEN (18). The fractions of mutant alleles in tumors were generally high, averaging 46% (with standard deviation (SD) of 18%). This is consistent with the expected early development of driver gene mutations during tumor evolution and the presence of non-neoplastic cells in all tumors, even macrodissected ones such as the samples used here. All mutations identified were confirmed to be absent in DNA from matched non-cancerous (“normal”) cells from each patient.

The presence of one of the mutations detected in each patient's tumor was then assessed in the CSF of the same patient using a sensitive, sequencing based method. This method reliably detects mutations with allele fractions as low as 0.01% (8, 19). An average of 4.8 mL of CSF (SD of 2.6) was collected from the 35 patients (Table 2). DNA could be purified from all CSF samples, though the amounts varied considerably (average of 417 ng, SD of 553 ng; Table 2). Primers were designed to amplify each of the 35 mutations as previously described (8, 19). Using this technology, we found that 74% of the 35 CSF samples contained detectable levels of tumor-derived DNA. The detectability of tumor DNA present in the CSF was not correlated with demographic characteristics, symptom duration, presence of hydrocephalus, contrast enhancement on imaging, or mutation type (Table 3a). The fraction of mutant alleles in the CSF was, as expected, usually lower than the fraction in the primary tumors, and was also much more variable than in the primary tumors. The average detectable mutant allele fraction in CSF was 12.2% (range 0.1% to 77%).

### Example 4

Relationship between mutations and clinical features. The great variation in mutant allele fraction among the CSF samples suggested that there might be some anatomical or biological factor underlying the differences. The tumors were distributed amongst the brain and spinal cord (Table 1) and malignancies arising in both organs were detected at similar frequencies (p=0.16; t-test). High-grade (WHO grade III-IV) tumors were more likely to have detectable CSF-tDNA than low-grade lesions (p=0.004; Table 3a), as evidenced by the fact that all but one high-grade tumor (18/19) was detected. The levels of CSF-tDNA were also higher in high-grade lesions than in low-grade lesions (mutant allele fractions of 16.3±21.2% vs. 2.8±6.8%). However, tumor size was not a statistically significant factor in predicting CSF-tDNA detectability or level (p=0.41; Table 3a).

Another important factor associated with CSF-tDNA levels was anatomic location. MRI scans were examined for the presence of contrast enhancement adjacent to a large CSF space (Table 1). Representative examples are provided in FIG. S1. Patients with lesions adjacent to a CSF reservoir in the brain or spinal cord were much more likely to have detectable levels of CSF-tDNA than the remaining lesions. Such reservoirs included the cortical surfaces, ventricles, as well as the basal and other cisterns. Accordingly, 86% of the 28 cases in which tumors were adjacent to CSF reservoir had detectable levels of CSF-tDNA. These cases included all 13 high-grade gliomas, all five ependymomas, and all five medulloblastomas that were in contact with the CSF. The four tumors in CSF contact that were not detectable were all low-grade gliomas. Moreover, none of the five patients whose tumors were entirely encapsulated by the brain or spinal cord parenchyma had detectable levels of CSF-tDNA (p<0.001, Table 3a). In addition, eighteen of the nineteen (˜95%) high-grade (WHO grade III or IV) tumors had detectable levels of CSF-tDNA. Upon multivariate logistic regression, only the location of tumors with respect to CSF and the tumor grade were statistically significant (Table 3b).

### Example 5

Genome-wide sequencing of DNA from the CSF. The results described above were performed after identifying at least one mutation in the primary tumor of each patient. In four patients with either brainstem or intramedullary spinal cord tumors, we also tested whether CSF-tDNA could be detected directly in their CSF via whole-exome sequencing (WES) without prior knowledge of the tumor genotype. These four samples were selected based on the critical and highly sensitive location of the malignancies, making surgery treacherous. We found that two of the four cases analyzed had levels of CSF-tDNA that were comparable to the levels identified through single amplicon sequencing when the same mutation was assessed (Table 4). Both detectable cases had greater than 10% mutant allele fractions in the CSF as measured by single amplicon sequencing. (Table 4). In contrast, the two cases in which WES was unable to identify CSF-tDNA had mutant allele fractions <1% as assessed by single amplicon sequencing (Table 4). As controls, we also performed WES on matched normal tissue and tumor tissues. The mutations were found in the tumors at a high frequency but were absent in normal tissues (Table 4).

### REFERENCES

The disclosure of each reference cited is expressly incorporated herein.
1. Ostrom Q T, et al. (2013) CBTRUS statistical report: Primary brain and central nervous system tumors diagnosed in the United States in 2006-2010. Neuro Oncol 15 Suppl 2: ii1-56.
2. Kros J M, et al. (2014) Circulating glioma biomarkers. Neuro Oncol.
3. Van Mieghem E, et al. (2013) Defining pseudoprogression in glioblastoma multiforme. European journal of neurology: the official journal of the European Federation of Neurological Societies 20 (10): 1335-1341.
4. Muller C, et al. (2014) Hematogenous dissemination of glioblastoma multiforme. Sci Transl Med 6 (247): 247ra101.
5. Macarthur K M, et al. (2014) Detection of brain tumor cells in the peripheral blood by a telomerase promoter-based assay. Cancer Res 74 (8): 2152-2159.
6. Sullivan J P, et al. (2014) Brain tumor cells in circulation are enriched for mesenchymal gene expression. Cancer Discov 4 (11): 1299-1309.
7. Diehl F, et al. (2008) Circulating mutant DNA to assess tumor dynamics. Nat Med 14 (9): 985-990.
8. Bettegowda C, et al. (2014) Detection of circulating tumor DNA in early- and late-stage human malignancies. Sci Transl Med 6 (224): 224ra224.
9. Dawson S J, et al. (2013) Analysis of circulating tumor DNA to monitor metastatic breast cancer. N Engl J Med 368 (13): 1199-1209.
10. Newman A M, et al. (2014) An ultrasensitive method for quantitating circulating tumor DNA with broad patient coverage. Nat Med 20 (5): 548-554.
11. Martignetti J A, et al. (2014) Personalized ovarian cancer disease surveillance and detection of candidate therapeutic drug target in circulating tumor DNA. Neoplasia 16 (1): 97-103.
12. Sidransky D, et al. (1992) Identification of ras oncogene mutations in the stool of patients with curable colorectal tumors. Science 256 (5053): 102-105.
13. Sidransky D, et al. (1991) Identification of p53 gene mutations in bladder cancers and urine samples. Science 252 (5006): 706-709.
14. Ralla B, et al. (2014) Nucleic acid-based biomarkers in body fluids of patients with urologic malignancies. Critical reviews in clinical laboratory sciences 51 (4): 200-231.
15. Hubers A J, Prinsen C F, Sozzi G, Witte B I, & Thunnissen E (2013) Molecular sputum analysis for the diagnosis of lung cancer. Br J Cancer 109 (3): 530-537.
16. Diehl F, et al. (2008) Analysis of mutations in DNA isolated from plasma and stool of colorectal cancer patients. Gastroenterology 135 (2): 489-498.
17. Kinde I, et al. (2013) Evaluation of DNA from the Papanicolaou test to detect ovarian and endometrial cancers. Sci Transl Med 5 (167): 167ra164.
18. Vogelstein B, et al. (2013) Cancer genome landscapes. Science 339 (6127): 1546-1558.
19. Kinde I, Wu J, Papadopoulos N, Kinzler K W, & Vogelstein B (2012) Detection and quantification of rare mutations with massively parallel sequencing. Proc Natl Acad Sci USA 108 (23): 9530-9535.
20. Woodworth G F, et al. (2013) Histopathological correlates with survival in reoperated glioblastomas. J Neurooncol 113 (3): 485-493.
21. Krueger D A, et al. (2010) Everolimus for subependymal giant-cell astrocytomas in tuberous sclerosis. N Engl J Med 363 (19): 1801-1811.
22. Pan W, Gu W, Nagpal S, Gephart M H, & Quake S R (2015) Brain tumor mutations detected in cerebral spinal fluid. Clin Chem 61 (3): 514-522.
23. von Hoff K & Rutkowski S (2012) Medulloblastoma. Current treatment options in neurology 14 (4): 416-426.
24. Bartlett F, Kortmann R, & Saran F (2013) Medulloblastoma. Clinical oncology 25 (1): 36-45.
25. Glass J P, Melamed M, Chernik N L, & Posner J B (1979) Malignant cells in cerebrospinal fluid (CSF): the meaning of a positive CSF cytology. Neurology 29 (10): 1369-1375.
26. Preusser M & Hainfellner J A (2012) CSF and laboratory analysis (tumor markers). Handbook of clinical neurology 104:143-148.
27. Allory Y, et al. (2014) Telomerase reverse transcriptase promoter mutations in bladder cancer: high frequency across stages, detection in urine, and lack of association with outcome. Eur Urol 65 (2): 360-366.
28. Destro A, et al. (2004) K-ras and p16 (INK4A) alterations in sputum of NSCLC patients and in heavy asymptomatic chronic smokers. Lung cancer 44 (1): 23-32.
29. Mack S C, et al. (2014) Epigenomic alterations define lethal CIMP-positive ependymomas of infancy. Nature 506 (7489): 445-450.
30. Gajjar A, Pfister S M, Taylor M D, & Gilbertson R J (2014) Molecular insights into pediatric brain tumors have the potential to transform therapy. Clin Cancer Res 20 (22): 5630-5640.
31. Rudin C M, et al. (2009) Treatment of medulloblastoma with hedgehog pathway inhibitor GDC-0449. N Engl J Med 361 (12): 1173-1178.
32. Thompson M C, et al. (2006) Genomics identifies medulloblastoma subgroups that are enriched for specific genetic alterations. J Clin Oncol 24 (12): 1924-1931.
33. Moreno L, et al. (2010) Utility of cerebrospinal fluid cytology in newly diagnosed childhood ependymoma. J Pediatr Hematol Oncol 32 (6): 515-518.
34. Weston C L, Glantz M J, & Connor J R (2011) Detection of cancer cells in the cerebrospinal fluid: current methods and future directions. Fluids and barriers of the CNS 8 (1): 14.
35. Khwaja F W, et al. (2007) Proteomic identification of biomarkers in the cerebrospinal fluid (CSF) of astrocytoma patients. J Proteome Res 6 (2): 559-570.
36. Roy S, et al. (2008) Protein biomarker identification in the CSF of patients with CNS lymphoma. J Clin Oncol 26 (1): 96-105.
37. Bougel S, et al. (2013) Methylation of the hTERT promoter: a novel cancer biomarker for leptomeningeal metastasis detection in cerebrospinal fluids. Clin Cancer Res 19 (8): 2216-2223.
38. Samuel N, Remke M, Rutka J T, Raught B, & Malkin D (2014) Proteomic analyses of CSF aimed at biomarker development for pediatric brain tumors. J Neurooncol 118 (2): 225-238.

39 Chamberlain M C, Kormanik P A, & Glantz M J (2001) A comparison between ventricular and lumbar cerebrospinal fluid cytology in adult patients with leptomeningeal metastases. Neuro Oncol 3 (1): 42-45.
40. Gajjar A, et al. (1999) Comparison of lumbar and shunt cerebrospinal fluid specimens for cytologic detection of leptomeningeal disease in pediatric patients with brain tumors. J Clin Oncol 17 (6): 1825-1828.
41. Thomas L, Di Stefano A L, & Ducray F (2013) Predictive biomarkers in adult gliomas: the present and the future. Curr Opin Oncol 25 (6): 689-694.
42. Olar A & Aldape K D (2014) Using the molecular classification of glioblastoma to inform personalized treatment. J Pathol 232 (2): 165-177.
43. Gajjar A J & Robinson G W (2014) Medulloblastoma-translating discoveries from the bench to the bedside. Nat Rev Clin Oncol.
44. Horn S, et al. (2013) TERT promoter mutations in familial and sporadic melanoma. Science 339 (6122): 959-961.
45. Huang F W, et al. (2013) Highly recurrent TERT promoter mutations in human melanoma. Science 339 (6122): 957-959.
46. Killela P J, et al. (2013) TERT promoter mutations occur frequently in gliomas and a subset of tumors derived from cells with low rates of self-renewal. Proc Natl Acad Sci USA 110 (15): 6021-6026.
47. Bettegowda C, et al. (2013) Exomic sequencing of four rare central nervous system tumor types. Oncotarget 4 (4): 572-583.
48. Kinde I, et al. (2013) TERT promoter mutations occur early in urothelial neoplasia and are biomarkers of early disease and disease recurrence in urine. Cancer Res 73 (24): 7162-7167.
49. Zhang M, et al. (2014) Somatic mutations of SUZ12 in malignant peripheral nerve sheath tumors. Nat Genet 46 (11): 1170-1172.

50 Agrawal N, et al. (2011) Exome Sequencing of Head and Neck Squamous Cell Carcinoma Reveals Inactivating Mutations in NOTCH1. Science.
51. Agrawal N, et al. (2012) Comparative Genomic Analysis of Esophageal Adenocarcinoma and Squamous Cell Carcinoma. Cancer Discov 2 (10): 899-905.
52. Bettegowda C, et al. (2011) Mutations in CIC and FUBPI contribute to human oligodendroglioma. Science 333 (6048): 1453-1455.

1. (canceled)

2. A method comprising:
a) receiving a Cerebral Spinal Fluid (CSF) sample from a human with a primary brain tumor, wherein said primary brain tumor is a glioma, a medulloblastoma, or an ependymoma and has been determined by imaging to abut a CSF reservoir, wherein the CSF reservoir is a ventricular reservoir or a basal cistern;
b) assaying tumor-specific DNA shed from said primary brain tumor in said CSF sample and determining the presence of one or more point mutations in said tumor-specific DNA; and
c) assaying nucleic acids from a normal tissue from the human and determining absence of the one or more point mutations determined to be present in said tumor-specific DNA;
wherein the one or more point mutations are present in one or more of TP53, IDH1, NF2, PIK3R1, PTCH1, and PTEN.

3. The method of claim 2, further comprising the step of assaying nucleic acids in a tissue sample from said primary brain tumor from the human and determining the one or more point mutations.

4. The method of claim 2, wherein the human has previously been subjected to surgical removal of the primary brain tumor.

5. The method of claim 2, wherein steps a) and b) are repeated using CSF samples collected from the human at different times.

6. The method of claim 3, wherein the step of assaying the nucleic acids in the tissue sample from said primary brain tumor is performed prior to the step of assaying said tumor-specific DNA in the CSF sample.

7. The method of claim 3, wherein the step of assaying the nucleic acids in the tissue sample from said primary brain tumor is performed using genome-wide screening.

8. The method of claim 3, wherein the step of assaying the nucleic acids in the tissue sample from said primary brain tumor is performed using exome-wide screening.

9. The method of claim 2, wherein the step of assaying in step b) employs a specific probe or a specific primer to detect the one or more point mutations, wherein the specific probe or the specific primer hybridizes at or within 200 nucleotides of the point mutation.

10. The method of claim 9, wherein the specific probe is employed.

11. The method of claim 9, wherein the specific primer is employed.

12. The method of claim 2, wherein nested amplification reactions are used to detect the one or more point mutations.

13. The method of claim 2, wherein the one or more point mutations comprise a point mutation in TERT promoter.

14. The method of claim 2, wherein the primary brain tumor is a glioma.

15. The method of claim 2, wherein the primary brain tumor is a medulloblastoma.

16. The method of claim 2, wherein the primary brain tumor is an ependymoma.
